(19) United States
(12) Patent Application Publication
(10) Pub. No.: US 2025/0166803 A1
(43) Pub. Date: May 22, 2025

(54) MACHINE-LEARNING-BASED WORKFLOW PLATFORM

(71) Applicant: Knowtex Inc., San Francisco, CA (US)

(72) Inventors: Caroline Chai Zhang, San Francisco, CA (US); Jocelyn Lillian Kang, San Francisco, CA (US)

(21) Appl. No.: 18/956,784

(22) Filed: Nov. 22, 2024

Related U.S. Application Data
(60) Provisional application No. 63/602,137, filed on Nov. 22, 2023.

(51) Int. Cl. G16H 40/20; G06Q 10/0633; G16H 50/20; G16H 70/20

(52) U.S. Cl. CPC G16H 40/20; G06Q 10/0633; G16H 50/20; G16H 70/20

(57) ABSTRACT

A machine learning-based clinical workflow system processes patient encounter data to generate structured data records and predicted diagnosis classification codes. The system may obtain an input record, generate vector embeddings, and identify reference records using vector similarity operations. A machine learning (ML) component may generate a structured data record based on the input and reference records. The system may also generate an ML instruction, identify reference codes, and produce a predicted code. The predicted code may include a predicted diagnosis classification code, a predicted procedural code, or a billing code. Vector databases storing embeddings of medical codes and records may facilitate efficient retrieval of relevant information. Some implementations may include prediction of billing codes to address revenue capture. Some implementations may utilize specialty-specific processes and data to enhance accuracy for particular medical fields. The system may incorporate clinician feedback to continuously improve performance and adapt to evolving healthcare practices.

### CROSS REFERENCE TO RELATED APPLICATIONS

This application claims priority to U.S. Application No. 63/602,137, titled “System and Method for Artificial Intelligence Powered Medical Notetaking and Clinical Workflows,” filed Nov. 22, 2023, which is hereby incorporated by reference in its entirety.

### FIELD

The present disclosure relates to clinical workflow management systems, and more particularly to a machine learning-based platform for automated medical documentation and coding using information retrieval augmentation.

### BRIEF DESCRIPTION OF THE DRAWINGS

This disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a block diagram of an example of a machine-learning-based workflow system.

FIG. 2 is a block diagram of an example internal configuration of a computing device.

FIG. 3 is a block diagram of an example of a clinical workflow platform.

FIG. 4 is a block schematic diagram showing an example of a machine-learning-based clinical workflow system.

FIGS. 5A and 5B are schematic diagrams illustrating example processes associated with a machine-learning-based clinical workflow.

FIG. 6 is a schematic diagram illustrating another example process associated with a machine-learning-based clinical workflow.

FIG. 7 is a schematic diagram illustrating another example process associated with a machine-learning-based clinical workflow.

FIG. 8 is a flow diagram illustrating an example of a technique associated with a machine-learning-based clinical workflow.

FIG. 9 is a flow diagram illustrating another example of a technique associated with a machine-learning-based clinical workflow.

FIG. 10 is a flow diagram illustrating another example of a technique associated with a machine-learning-based clinical workflow.

FIG. 11 is a flow diagram illustrating another example of a technique associated with a machine-learning-based clinical workflow.

FIGS. 12A-12D illustrate example interfaces for displaying and managing medical coding information in a clinical workflow system.

### DETAILED DESCRIPTION

Various aspects of the disclosure are described more fully hereinafter with reference to the accompanying drawings. This disclosure may, however, be embodied in many different forms and are not to be construed as limited to any specific structure or function presented throughout this disclosure. Rather, these aspects are provided so that this disclosure will be thorough and complete, and will fully convey the scope of the disclosure to those skilled in the art. One skilled in the art may appreciate that the scope of the disclosure is intended to cover any aspect of the disclosure disclosed herein, whether implemented independently of or combined with any other aspect of the disclosure. For example, an apparatus may be implemented or a method may be practiced using any quantity of the aspects set forth herein. In addition, the scope of the disclosure is intended to cover such an apparatus or method which is practiced using other structure, functionality, or structure and functionality in addition to or other than the various aspects of the disclosure set forth herein. Any aspect of the disclosure disclosed herein may be embodied by one or more elements of a claim.

Aspects and examples generally include a method, apparatus, network node, system, computer program product, non-transitory computer-readable medium, computing device, and/or processing system as described or substantially described herein with reference to and as illustrated by the drawings and specification.

This disclosure may be readily utilized as a basis for modifying or designing other structures for carrying out the same purposes of the present disclosure. Such equivalent constructions do not depart from the scope of the appended claims. Characteristics of the concepts disclosed herein, both their organization and method of operation, together with associated advantages, are better understood from the following description when considered in connection with the accompanying figures. Each of the figures is provided for the purposes of illustration and description, and not as a definition of the limits of the claims.

While aspects are described in the present disclosure by illustration to some examples, such aspects may be implemented in many different arrangements and scenarios. Techniques described herein may be implemented using different platform types, devices, systems, shapes, sizes, and/or packaging arrangements. For example, some aspects may be implemented via integrated chip embodiments or other non-module-component-based devices (e.g., end-user devices, communication devices, computing devices, industrial equipment, retail/purchasing devices, medical devices, and/or artificial intelligence devices). Aspects may be implemented in chip-level components, modular components, non-modular components, non-chip-level components, device-level components, and/or system-level components. Devices incorporating described aspects and features may include additional components and features for implementation and practice of claimed and described aspects. For example, transmission and reception of signals or data may include one or more components for analog and digital purposes (e.g., hardware components including antennas, radio frequency (RF) chains, power amplifiers, modulators, buffers, processors, interleavers, adders, and/or summers). Aspects described herein may be practiced in a wide variety of devices, components, systems, distributed arrangements, and/or end-user devices of varying size, shape, and constitution.

Several aspects of a machine-learning-based clinical workflow system will now be presented with reference to various apparatuses and techniques. These apparatuses and techniques will be described in the following detailed description and illustrated in the accompanying drawings by various blocks, modules, components, circuits, steps, processes, or algorithms (collectively referred to as “elements”). These elements may be implemented using hardware, software, or a combination of hardware and software. Whether such elements are implemented as hardware or software depends upon the particular application and design constraints imposed on the overall system.

In the rapidly evolving landscape of healthcare technology, medical professionals face challenges in efficiently keeping digital records of patient encounters and accurately coding diagnoses and procedures. The traditional approach to clinical documentation often involves time-consuming manual processes, where healthcare providers must transcribe conversations, compose detailed notes, and select appropriate medical codes. This manual workflow not only diverts valuable time away from patient care but also introduces the potential for errors and inconsistencies in documentation and coding. The complexity of medical terminology, coupled with frequent updates to coding systems such as International Classification of Diseases, Tenth Revision (referred to as “ICD-10 codes” or “ICD-10-CM codes”) and current procedural terminology (CPT) codes, further compounds these challenges, making it difficult for healthcare providers to stay current and select the most appropriate codes for each patient encounter.

The increasing volume of patient data and the need for more detailed documentation to support value-based care models have intensified the administrative burden on healthcare providers. Clinicians often struggle to balance the demands of thorough documentation with the need to maintain meaningful patient interactions. This tension can lead to delayed or incomplete documentation, potentially impacting the quality of patient care and the accuracy of medical records. Furthermore, the lack of standardization in documentation practices across different healthcare settings and specialties can result in variability in the quality and completeness of clinical notes, making it challenging to ensure consistent, high-quality care and accurate billing.

Existing solutions for automated medical documentation and coding have limitations in their ability to adapt to the nuances of different medical specialties and individual provider preferences. Many systems rely on rigid templates or rule-based approaches that may not capture the full complexity of patient encounters. Additionally, the challenge of integrating automated documentation and coding systems with existing electronic health record (EHR) platforms presents a hurdle for healthcare organizations seeking to streamline their workflows. These technical challenges underscore the need for more sophisticated, adaptive solutions that can seamlessly integrate into clinical workflows, improve documentation accuracy, and enhance the overall efficiency of healthcare delivery.

Implementations of this disclosure address problems such as these by providing a machine-learning-based (ML-based) workflow platform. The ML-based workflow platform may facilitate automated medical coding and documentation. In some implementations, the system may be configured to receive an input record (which may be referred to as a “transcript”) associated with a doctor-patient interaction. The input record may be a text record, an audio recording, or a video recording, among other examples. The ML-based workflow platform may generate, using a machine learning (ML) component and based on the input record and at least one reference record, a structured data record (SDR) associated with the interaction. The SDR may include any type of SDR such as, for example, a template-based information record, a summary, a chart, and/or a note, among other examples. This SDR, which may be referred to as a SOAP (Subjective, Objective, Assessment, Plan) note, may be provided to a service engine system configured to generate, based on the SDR, a predicted diagnosis classification code such as a code used to classify medical diagnosis for billing purposes, charting purposes, or the like. The code may be, for example, an ICD-10 codes, a CPT code, or any other standardized code used in healthcare for classification, billing, or research purposes, among other examples.

An ML component refers to software capable of performing ML. ML is a subset of artificial intelligence (AI) that involves the development of algorithms and statistical models enabling computers to perform tasks without explicit programming. ML leverages large datasets to identify patterns, make decisions, and improve over time based on experience. ML focuses on creating systems that can learn from data, adapt to new inputs, and generate predictions or actions.

For example, an ML component may be or include one or more ML models, ML algorithms, and/or ML systems including combinations of ML algorithms and ML models. An ML component may be implemented on any number of different hardware devices and may include one or more machine learning models. ML is a field of study that gives computers the ability to perform certain tasks without being explicitly programmed to perform those tasks. ML explores the study and construction of algorithms, also referred to herein as tools, models, and/or components, which may learn from existing data and make predictions about new data. Such ML tools operate by building a model from example training data in order to make data-driven predictions or decisions expressed as outputs or assessments. Although example embodiments are presented with respect to a few ML models, the principles presented herein may be applied to other ML models. In some example embodiments, different ML models may be used. ML models may include, for example, K-means clustering models, linear regression models, Logistic Regression (LR) models, Naive-Bayes models, Random Forest (RF) regression models, gradient boost models, neural networks (NN), matrix factorization models, and/or Support Vector Machines (SVMs).

Some implementations include an embedding engine configured to generate vector embeddings from an input record and associated metadata. These vector embeddings may be used by a matching engine to query a vector database and retrieve relevant historical information. The matching engine may filter this information to produce a set of reference records that may be most applicable to a current input record. The ML component may use these reference records along with the input record to generate the SDR. As used herein, the term “reference record” includes, but is not limited to, previously generated SDRs, clinical notes, curated examples of best practices, or any other structured clinical documentation that can serve as a guide for generating new records.

Some implementations include a feedback mechanism that allows for continuous improvement. After the SDR is generated, it may be presented to a clinician through an interface for review and potential editing. The edited SDR may then processed to create new vector embeddings, which may be stored in the vector database for future use. This feedback loop allows the system to learn from each interaction and adapt to individual clinician preferences and institutional guidelines. As used herein, the term “vector embedding” refers to a vector representation that captures the semantic meaning of text in a high-dimensional space.

Some implementations include mechanisms for handling new codes and adapting to changes in coding standards. When a new code is introduced, it may be processed by the embedding engine to create new vector embeddings, which may be added to the vector database. In this way, some implementations facilitate remaining up-to-date with the latest coding standards and adapting to changes in medical terminology and classification.

As indicated above, implementations may incorporate or employ one or more AI or ML (collectively referred to as AI/ML) systems utilizing one or more models trained for designated purposes. The use or incorporation of such AI/ML systems, particularly for the deployment of specific features or functions, may be disabled by default, necessitating that a user, an organization, or both actively opt in to activate and utilize these features or functions that leverage AI/ML systems. Consent for employing the AI/ML systems or specific features thereof may be obtained through various mechanisms, such as explicit user permissions granted prior to the engagement with an AI/ML feature, administrative approvals configured via administrator settings, or a combination thereof. Upon obtaining such consent, users may receive notifications regarding their interaction with AI/ML systems or features, such notifications being conveyed through electronic messages (e.g., via chat, email services, or within client applications or web interfaces) or by on-screen prompts, which may be presented on a per-interaction basis. Additionally, these users may be provided with a streamlined process to rescind their consent, such as through a form or similar element accessible within a client application, webpage, or on-screen prompt, allowing individual users to opt out from further interactions with the AI/ML systems or features.

To enhance user privacy and security, and to provide additional advantages, the AI/ML processing system may be restricted from employing personal information of users or organizations (e.g., audio, video, chat, screen-sharing content, attachments, or similar communication-type data, such as polling results, whiteboards, or reactions) for the purpose of training AI/ML models. Instead, personal information may be used solely for inference operations within the AI/ML processing system. The training of AI/ML models may be conducted using one or more commercially licensed datasets that exclude user or organizational personal information, thus isolating the training data from identifiable user data to preserve privacy.

The implementations of this disclosure present and relate to improvements in computing technology which aggregates and normalizes clinical data, diagnostic coding data, and procedural coding data across multiple disparate digital information sources from non-standardized formats into a standardized format usable with a cross-pipeline ontology of a server environment associated with multiple machine learning pipelines. This enables the processing and exchange of clinical data, diagnostic coding data, procedural coding data, and related attribute data in a standardized format usable for each of the multiple machine learning pipelines that connect to the server environment. Moreover, the implementations of this disclosure present and relate to technology distinct from mere commercial activity and which cannot be practiced in the human mind at least in that they address various aspects rooted in computing technology, for example, text and audio feature extraction processing using trained machine learning models, aggregated feature normalization from non-standardized formats to a standardized format, generation and storage of embedding vectors, and the like. The implementations of this disclosure are further necessarily rooted in computing technology at least by their focus on electronic clinical workflow applications and the software systems that implement them over networks such as the Internet.

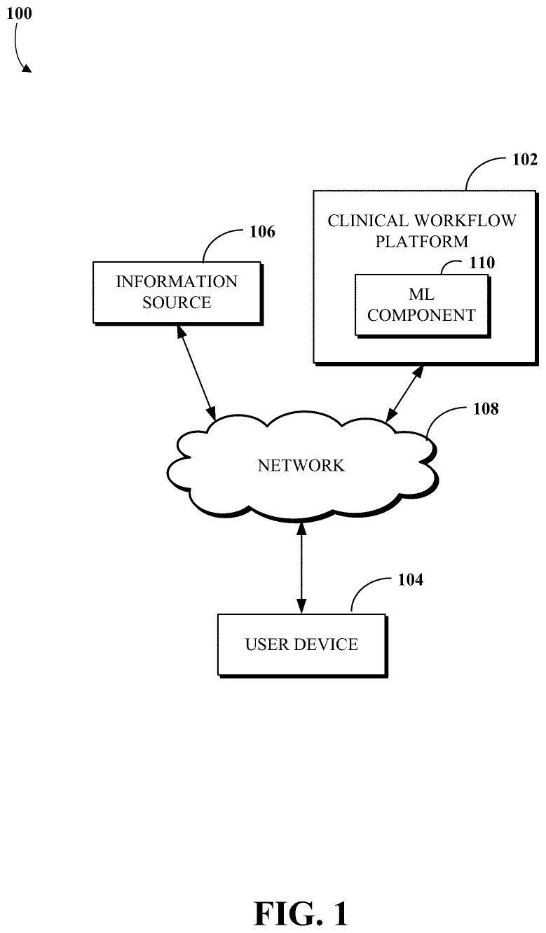

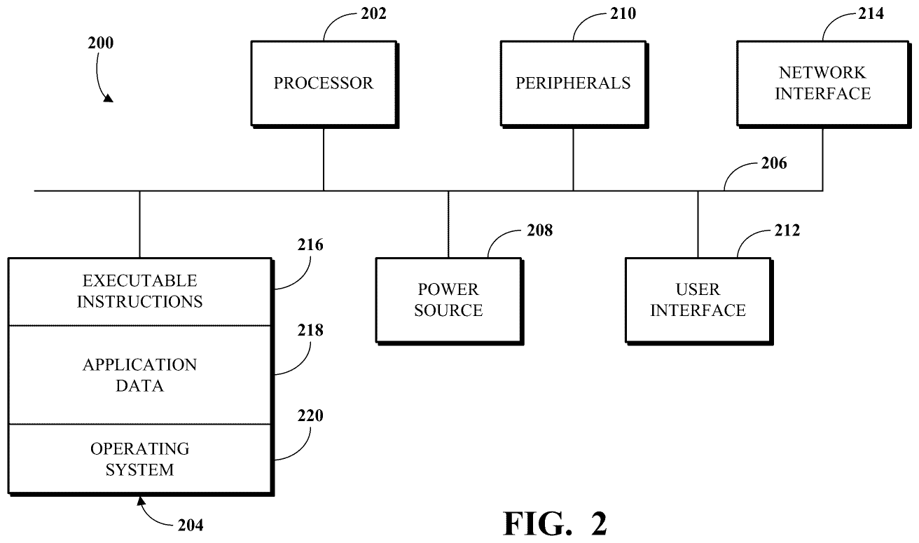

FIG. 1 is a block diagram of an example of a machine-learning-based clinical workflow system 100, which can be or include a distributed computing system, a cloud computing system, and/or a clustered computing system, among other examples. As shown, the system 100 includes a clinical workflow platform 102, a user device 104, and an information source 106, communicatively coupled by a network 108. Although one clinical workflow platform 102, one user device 104, and one information source 106 are shown in FIG. 1, multiple clinical workflow platforms 102, multiple user devices 104, and/or multiple information sources 106 can be included in the system 100. Moreover, although the clinical workflow platform 102 is shown in a single computing device, in some cases the clinical workflow platform 102, the user device 104, and/or the information source 106 can include respective one or more computing devices, communicatively coupled to one another. The computing devices in the clinical workflow platform 102, the user device 104, and/or the information source 106 can include any or all of the features shown and described in connection with FIG. 2.

The clinical workflow platform 102 can include, interface with, and/or otherwise access the user device 104, the information source 106, and an ML component 110. As indicated above, an ML component (e.g., the ML component 110) may be or include one or more ML models, ML algorithms, and/or ML systems including combinations of ML algorithms and ML models. An ML component may be implemented on any number of different hardware devices and may include one or more machine learning models, ML algorithms, and ML systems described herein. For example, although the ML component 110 is shown as being part of the clinical workflow platform 102 in FIG. 1, the ML component 110 may be implemented on other devices, such as the user device 104, the information source 106, an additional computing device, multiple computing devices (e.g., a distributed computing system), and/or any combination of the clinical workflow platform 102, the user device 104, the information source 106, and/or an additional computing device. The user device 104, the information source 106, and/or an ML component 110 may be communicatively and/or operatively coupled to the clinical workflow platform 102. For example, the user device 104, the information source 106, and/or an ML component 110 may be communicatively coupled via the network 108.

The clinical workflow platform 102 may provide a framework for generating a clinical workflow that can be initiated via one or more user devices, such as the user device 104. The user device 104 may include one or more user devices, which may include any computing device that can communicate with the clinical workflow platform 102 using the network 108. The user device 104 may be utilized by one or more users. The user device 104 may include one or more computing devices such as personal computers (PCs), laptops, mobile phones, smart phones, tablet computers, netbook computers, network connected home appliances, and/or any other computing devices, such as the computing device shown in FIG. 2. The user device 104 may include one or more user applications, such as a web browser application (e.g., a web browser) or a native application (e.g., a mobile application), to interact with the clinical workflow platform 102. For example, the user device 104 may execute the web browser application to receive information from (e.g., to provide input to the clinical workflow platform 102 and/or to receive output from the clinical workflow platform 102) the clinical workflow platform 102 and/or the ML component 110, such as a graphical user interface output from the clinical workflow platform 102 and/or ML component 110. As an example, a user application (e.g., a web browser, a mobile application, etc.) may enable a user to interact with the clinical workflow platform 102 and/or the ML component 110 by providing input to the clinical workflow platform 102 and/or the ML component 110 via a graphical user interface generated by the user device 104.

The clinical workflow platform 102 may obtain information from and/or provide information to the information source 106 via the network 108 using a communication interface. The information source 106 may include one or more of cloud storage services, object or file hosting services, services that store structured or unstructured data, and/or any other remote data sources. The clinical workflow platform 102 and/or the information source 106 may store, organize, and manage data in one or more databases or data structures. The information source 106 may be connected directly to the clinical workflow platform 102, the user device 104, and/or the ML component 110 or indirectly by way of one or more additional devices. In some cases, the information source 106 may be included in the clinical workflow platform 102, the user device 104, and/or the ML component 110. The information source 106 may communicate with the ML component 110 via the clinical workflow platform 102.

In some implementations, information source 106 may provide one or more reference records or reference codes pertaining to a particular subject or subject matter (e.g., a medical subject, clinical subject, and/or other subject), such as a patient, a disease or disease state, a procedure, a product, an activity, a topic, a code set (e.g., ICD and/or CPT code sets), a code description (e.g., a ICD code or CPT code description), a medical guideline, a code exclusion, a synonym, a disease name, and/or a diagnosis code (e.g., an ICD-10 diagnostic code). For example, the reference records or reference codes may indicate, for the subject matter, medical coding information associated with one or more of (i) billing, (ii) diagnostic, (iii) procedure, (iv) facility information, (v) code description, (vi) language information, (vii) a medical guideline, (viii) a code exclusion, (ix) a synonym, and/or (x) a disease name, among other reference records or reference codes indicated by the information source 106. Thus, for each subject matter, the information source 106 may include a plurality of reference records or reference codes that correspond to different types of billing, diagnosis, procedure, facility information, code description, and/or language information. In some implementations, the information source may be implemented by a third-party service that provides reference records or reference codes. In some implementations, the information source may be included in (e.g., integrated with) the clinical workflow platform 102, the user device 104, and/or the ML component 110.

The network 108 may be a public communication network (e.g., the Internet, cellular data network, dialup connectivity, etc.), a private communications network (e.g., private LAN, leased line, etc.), or a combination of a public communications network and a private communications network. In some cases, the network 108 may include, and/or may communicate with, any one or more of a Bluetooth network, a near-field communication network, a satellite communication network, a wireless communication network, and/or any other communication network. The network 108 may include any combination of wired and/or wireless networks. The communication over the network 108 can traverse one or more of the Internet, a wide area network (WAN), a metropolitan area network (MAN), a local area network (LAN), a virtual private network (VPN), a wireless local area network (WLAN), a virtual private wireless network (VP), a radio access network, a mobile data network, a power distribution network, a satellite network, a plain old telephone system (POTS), and/or a cellular or third generation (3G) or fourth generation (4G) data network, among other examples, and/or any combination of the above.

FIG. 2 is a block diagram of an example internal configuration of a computing device 200. In some implementations, the computing device 200 may implement one or more of the clinical workflow platform 102, the user device 104, or the information source 106 of the system 100 shown in FIG. 1.

The computing device 200 includes components or units, such as a processor 202, a memory 204, a bus 206, a power source 208, peripherals 210, a user interface 212, a network interface 214, other suitable components, or a combination thereof. One or more of the memory 204, the power source 208, the peripherals 210, the user interface 212, or the network interface 214 can communicate with the processor 202 via the bus 206.

The processor 202 is a central processing unit, such as a microprocessor, and can include single or multiple processors having single or multiple processing cores. Alternatively, the processor 202 can include another type of device, or multiple devices, configured for manipulating or processing information. For example, the processor 202 can include multiple processors interconnected in one or more manners, including hardwired or networked. The operations of the processor 202 can be distributed across multiple devices or units that can be coupled directly or across a local area or other suitable type of network. The processor 202 can include a cache, or cache memory, for local storage of operating data or instructions.

The memory 204 includes one or more memory components, which may each be volatile memory or non-volatile memory. For example, the volatile memory can be random access memory (RAM) (e.g., a DRAM module, such as DDR SDRAM). In another example, the non-volatile memory of the memory 204 can be a disk drive, a solid state drive, flash memory, or phase-change memory. In some implementations, the memory 204 can be distributed across multiple devices. For example, the memory 204 can include network-based memory or memory in multiple clients or servers performing the operations of those multiple devices.

The memory 204 can include data for immediate access by the processor 202. For example, the memory 204 can include executable instructions 216, application data 218, and an operating system 220. The executable instructions 216 can include one or more application programs, which can be loaded or copied, in whole or in part, from non-volatile memory to volatile memory to be executed by the processor 202. For example, the executable instructions 216 can include instructions for performing some or all of the techniques of this disclosure. The application data 218 can include user data, database data (e.g., database catalogs or dictionaries), or the like. In some implementations, the application data 218 can include functional programs, such as a web browser, a web server, a database server, another program, or a combination thereof. The operating system 220 can be, for example, Microsoft Windows®, Mac OS X®, or Linux®; an operating system for a mobile device, such as a smartphone or tablet device; or an operating system for a non-mobile device, such as a mainframe computer.

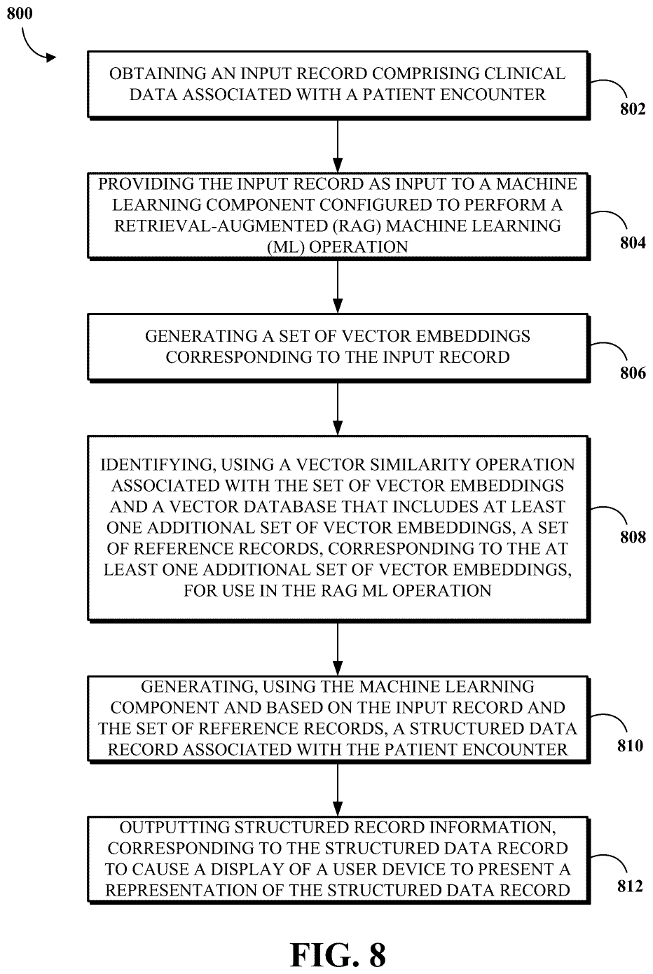

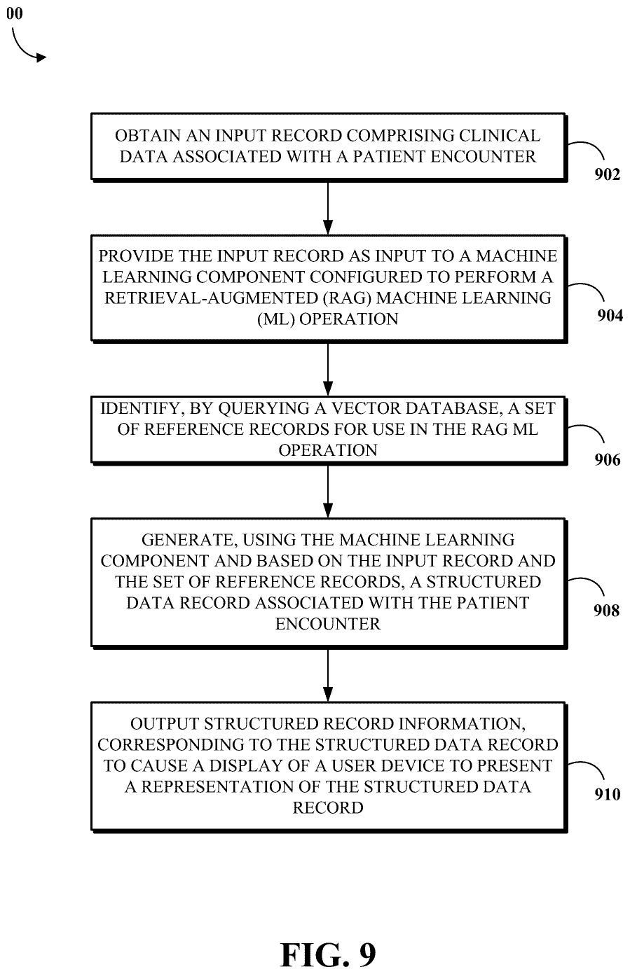

The processor 202 may implement one or more techniques or perform one or more operations associated with machine-learning based clinical workflows, as described in more detail elsewhere herein. For example, the processor 202 may perform or direct operations of, for example, technique 800 of FIG. 8, technique 900 of FIG. 9, or other techniques as described herein (alone or in conjunction with one or more other processors). The memory 204 may store data and program codes for the computing device 200. In some examples, the memory 204 may include a non-transitory computer-readable medium storing a set of instructions (for example, code or program code). The memory 204 may include one or more memories, such as a single memory or multiple different memories (of the same type or of different types). For example, the set of instructions, when executed (for example, directly, or after compiling, converting, or interpreting) by the processor 202, may cause the processor to cause the computing device 200 to perform technique 800 of FIG. 8, technique 900 of FIG. 9, or other techniques as described herein. In some examples, executing instructions may include running the instructions, converting the instructions, compiling the instructions, and/or interpreting the instructions, among other examples.

The power source 208 provides power to the computing device 200. For example, the power source 208 can be an interface to an external power distribution system. In another example, the power source 208 can be a battery, such as where the computing device 200 is a mobile device or is otherwise configured to operate independently of an external power distribution system. In some implementations, the computing device 200 may include or otherwise use multiple power sources. In some such implementations, the power source 208 can be a backup battery.

The peripherals 210 includes one or more sensors, detectors, or other devices configured for monitoring the computing device 200 or the environment around the computing device 200. For example, the peripherals 210 can include a geolocation component, such as a global positioning system location unit. In another example, the peripherals can include a temperature sensor for measuring temperatures of components of the computing device 200, such as the processor 202. In some implementations, the computing device 200 can omit the peripherals 210.

The user interface 212 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, or other suitable display.

The network interface 214 provides a connection or link to a network (e.g., the network 108 shown in FIG. 1). The network interface 214 can be a wired network interface or a wireless network interface. The computing device 200 can communicate with other devices via the network interface 214 using one or more network protocols, such as using Ethernet, transmission control protocol (TCP), internet protocol (IP), power line communication, an IEEE 802.X protocol (e.g., Wi-Fi, Bluetooth, or ZigBee), infrared, visible light, general packet radio service (GPRS), global system for mobile communications (GSM), code-division multiple access (CDMA), Z-Wave, another protocol, or a combination thereof.

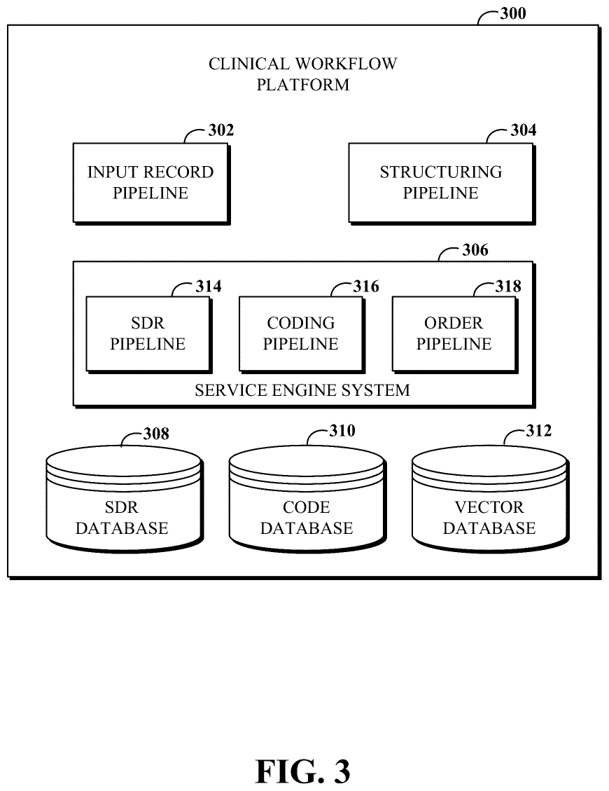

FIG. 3 is a block diagram of an example of a clinical workflow platform 300. The clinical workflow platform 300 may be, be similar to, include, or be included in the clinical workflow platform 102 shown in FIG. 1. As shown, the clinical workflow platform 300 includes an input record pipeline 302, a structuring pipeline 304, a service engine system 306, an SDR database 308, a code database 310, and a vector database 312.

The clinical workflow platform 300 may provide a comprehensive solution for automating and streamlining medical documentation and coding processes, addressing the challenges faced by healthcare providers in efficiently managing patient encounters. As shown in FIG. 3, the clinical workflow platform 300 comprises several interconnected components that work together to transform raw clinical data into structured, coded information suitable for electronic health records and billing purposes.

The input record pipeline 302 serves as the entry point for clinical data associated with patient encounters. This pipeline is designed to handle various types of input records, including text data from written notes, audio data from recorded conversations between healthcare providers and patients, and even video data from telemedicine sessions. The input record pipeline 302 processes these diverse data types and prepares them for further analysis and structuring. The input record may be generated by the user device 104 of FIG. 1, or from another service or component.

The input record pipeline 302 may be or include an audio pipeline, a textual pipeline, a video pipeline, or any combination of these. For example, the input record pipeline 302 may be or include a combination of a speech recognition pipeline and a transcription pipeline. In some implementations, the input record pipeline 302 may automatically determine whether the input record is an audio input record or a textual input record. If the input record is an audio input record, the input record will be transmitted to the speech recognition pipeline for processing. If the input record is a textual input record, the input record pipeline 302 will transmit the textual input record to the transcription pipeline for processing.

The input record pipeline 302 may be capable of processing various types of input records, providing flexibility and adaptability to different clinical scenarios and data sources. For example, the input record may include handwritten clinical notes scanned and digitized using optical character recognition (OCR) technology, voice recordings of patient-doctor conversations captured during in-person visits, video recordings from telemedicine sessions including both audio and visual information, structured data entered directly into electronic health record (EHR) systems, text messages or chat logs from patient-provider communication platforms, email correspondence between healthcare professionals discussing patient cases, dictated notes transcribed by medical transcription services, data from wearable devices or remote patient monitoring systems, scanned images of medical documents such as lab reports or referral letters, or social media posts or messages related to patient health concerns (with appropriate consent).

The ability of the input record pipeline 302 to handle diverse input record types may offer several potential advantages. It may allow for improved data capture by accepting various input formats, potentially leading to more comprehensive documentation of patient encounters. The system may provide enhanced workflow flexibility, allowing healthcare providers to choose convenient data entry methods based on preferences or specific situations. There may be opportunities for seamless integration with existing clinical workflows and technologies. The system's flexibility in accepting diverse inputs may make it suitable for use across various medical specialties with different documentation needs. As new technologies and data sources emerge in healthcare, the system may be well-positioned to incorporate these innovations. The ability to process multiple input types may promote more inclusive documentation practices. By allowing input from multiple sources, the system may facilitate cross-verification of information. Additionally, the system's capability to handle diverse inputs may support better communication and information sharing among care team members, potentially improving coordination of care.

The input record pipeline 302 is connected to the structuring pipeline 304, which plays a role in converting the raw input data into a structured format. The structuring pipeline 304 may employ advanced natural language processing techniques and machine learning algorithms to extract relevant clinical information from the input records. The structuring pipeline 304 may be configured to identify elements of a patient encounter, such as symptoms, diagnoses, treatments, and follow-up plans, and organize them into a standardized SDR. The SDR format may allow for easy retrieval, analysis, and integration with other healthcare systems.

In some implementations, the SDR may include a SOAP note. SOAP notes may provide a structured approach for documenting patient encounters in healthcare settings. SOAP is an acronym that stands for Subjective, Objective, Assessment, and Plan. This format may allow healthcare providers to organize and present clinical information in a clear and standardized manner.

The components of a SOAP note may include a subjective component. This component may contain information reported by the patient, their chief complaint, symptoms, or relevant history. For example, “Patient reports severe headache for the past 3 days, describes pain as throbbing and localized to the right temple.” The components of a SOAP note also may include an objective component. The objective component may include observable and measurable data collected by the healthcare provider. For example, “Blood pressure 130/80 mmHg, temperature 98.6° F., pupils equal and reactive to light, no visible signs of head trauma.” The components of a SOAP note may include an assessment component. The assessment component ay contain the healthcare provider's analysis and interpretation of the subjective and objective information. For example, “Suspected tension headache, possibly triggered by recent stress at work.” The components of a SOAP note may include a plan component. The plan component may outline the treatment plan, and may include medications, tests, referrals, or follow-up instructions. For example, “Prescribed ibuprofen 400 mg every 6 hours as needed for pain, recommended stress reduction techniques, follow-up in 2 weeks if symptoms persist.”

In some implementations, the structuring pipeline 304 could utilize deep learning models to improve extraction accuracy. Alternatively, it may incorporate rule-based systems alongside machine learning for a hybrid approach. In some implementations, the clinical workflow platform may include a retrieval-augmented generation (RAG) machine learning component. The RAG ML component may combine the strengths of large language models with the ability to access and utilize external knowledge sources. This approach may allow the system to generate more accurate and contextually relevant SDRs and medical codes. The RAG ML component may operate by first retrieving relevant information from a knowledge base, such as the vector database 312 or other specialized medical databases. This retrieval process may use vector similarity searches to identify the most pertinent reference documents, clinical guidelines, or historical cases related to the current patient encounter.

Once relevant information is retrieved, the RAG ML component may use this external knowledge to augment its generation process. For example, when creating an SDR, the RAG ML component may incorporate specific medical terminology, standard formatting, or relevant clinical guidelines that were retrieved from the knowledge base. This may result in SDRs that are not only structurally correct but also align with current medical best practices. In the context of medical coding, the RAG ML component may use retrieved information about coding guidelines, recent updates to coding systems, and similar historical cases to inform its code predictions. This may lead to more accurate and justifiable code assignments, potentially reducing coding errors and improving billing accuracy.

The RAG approach may be particularly beneficial in handling rare or complex cases where the base model's training data may be limited. By accessing external knowledge, the system may be able to provide more informed and reliable outputs even for uncommon medical scenarios. In some implementations, the RAG ML component may include a feedback loop that allows it to learn from each interaction. As clinicians review and potentially modify the generated SDRs or predicted codes, this feedback may be used to refine the retrieval process, adjusting the relevance scores of different knowledge sources for future queries. The RAG ML component may also be designed to handle multi-modal inputs. For instance, it may be able to process textual data from clinical notes alongside visual data from medical imaging or numerical data from lab results. This multi-modal capability may allow for a more comprehensive understanding of the patient's condition, potentially leading to more accurate SDRs and code predictions.

In some cases, the RAG component may be implemented using a combination of transformer-based language models for generation and dense retrieval models for information lookup. This architecture may allow for efficient scaling of the knowledge base without significantly increasing inference time. The RAG approach may also enhance the explainability of the system's outputs. By explicitly referencing the external knowledge used in generation, the system may provide clinicians with clear rationales for its SDR structures or code predictions, potentially increasing trust and adoption among healthcare providers.

The service engine system 306 is a component of the clinical workflow platform 300 that may perform various retrieval-augmented machine learning operations on the structured data. This system includes multiple specialized engines, such as an SDR pipeline 314, a coding pipeline 316, and an order pipeline 318. The SDR pipeline 314 may further refine and enhance the structured data records, ensuring they meet specific formatting and content requirements. The coding pipeline 316 may be tasked with generating appropriate medical codes, such as ICD-10 diagnostic codes and CPT procedure codes, based on the information contained in the SDRs. The order pipeline 318 may facilitate the generation of medical orders, such as prescriptions, lab tests, or imaging studies, as indicated by the clinical data. In some cases, the service engine system 306 could include additional pipelines for tasks like clinical decision support or patient risk stratification.

To support its operations, the clinical workflow platform 300 incorporates several databases. In some implementations, the SDR database 308, code database 310, and vector database 312 may each represent a single database or multiple interconnected databases within a larger data store. The choice of database type may depend on the specific requirements of the data being stored and the operations being performed. For example, relational databases may be used for structured data with well-defined relationships, such as patient records or medical codes. NoSQL databases may be employed for handling large volumes of unstructured or semi-structured data, which may be particularly useful for storing and querying diverse input records. Time-series databases may be utilized for efficiently managing and analyzing temporal data, such as patient vitals or medication schedules. Graph databases may be implemented to represent and query complex relationships between different clinical entities. In some cases, a hybrid approach combining multiple database types may be used to optimize performance and flexibility. The databases may be distributed across multiple servers or cloud platforms to enhance scalability, reliability, and data accessibility. Additionally, the system may employ data virtualization techniques to present a unified view of data stored across different physical locations or database systems.

The SDR database 308 serves as a repository for structured data records, allowing for storage and retrieval of patient encounter information. This database may store current SDRs and maintain historical records, which can be valuable for tracking patient progress over time and identifying trends in healthcare delivery. In some implementations, the SDR database 308 could utilize a distributed storage system for improved scalability and redundancy.

The code database 310 is a component that contains information about medical coding systems, including ICD-10 and CPT codes. This database may be regularly updated to reflect changes in coding standards, helping to ensure that the platform generates up-to-date and accurate codes. The code database 310 may include not only the codes themselves but also associated metadata such as code descriptions, usage guidelines, and exclusion criteria, which can be helpful for proper code selection. In some aspects, the code database 310 could incorporate machine learning models to suggest code updates based on emerging medical practices.

The vector database 312 stores vector embeddings. Vector embeddings are numerical representations of data in a high-dimensional space that capture semantic relationships and contextual information. The vector embeddings in the vector database 312 may include, for example, high-dimensional representations of input records, clinical concepts, SDRs, or medical codes, among other examples. The vector database 312 may enable sophisticated similarity searches and information retrieval operations, which may facilitate the platform's retrieval-augmented machine learning capabilities. By leveraging vector embeddings, the platform may quickly identify relevant historical records, similar cases, or appropriate codes based on the context of the current patient encounter. In some implementations, the vector database 312 may use techniques like locality-sensitive hashing for faster similarity searches in high-dimensional spaces.

In the context of clinical workflows, vector embeddings may be generated from various types of medical data, such as patient records, clinical notes, or medical codes. These embeddings may preserve the semantic meaning of the original data while allowing for efficient computational processing and comparison. For example, vector embeddings of medical terms may position similar concepts closer together in the vector space, enabling the system to identify related medical concepts even if they are described using different terminology.

Vector databases offer several advantages and functionalities that may enhance the performance and capabilities of clinical workflow systems. These databases may be optimized for storing and querying high-dimensional vector data, allowing for rapid similarity searches and efficient retrieval of relevant information. In some implementations, vector databases may support approximate nearest neighbor (ANN) search algorithms, which can significantly speed up similarity queries in large datasets. This capability may be particularly useful in clinical settings where quick access to relevant historical cases or similar patient profiles is crucial for decision-making. Additionally, vector databases may offer features such as real-time indexing, allowing new data to be immediately available for queries, and support for multi-modal data, enabling the system to work with diverse types of medical information, including text, images, and numerical data.

The vector embeddings used in the system may incorporate specialty-specific context vectors that capture the unique terminology, procedures, and diagnostic patterns associated with different medical specialties. For example, in oncology, the embeddings may encode relationships between cancer types, staging information, and treatment protocols, while in cardiology, the embeddings may capture associations between heart conditions, ECG findings, and cardiovascular interventions. This specialty-aware representation may allow the system to provide more accurate and relevant suggestions based on the specific medical domain of the patient encounter.

The system may integrate patient history information into the vector embeddings by considering temporal relationships and progression patterns in the patient's medical timeline. The embeddings may capture how conditions evolve over time, how treatments affect outcomes, and how different aspects of the patient's medical history relate to current symptoms or diagnoses. For instance, the vector representations may encode information about previous procedures, medication responses, and chronic condition management, allowing the system to consider this historical context when generating new structured data records or suggesting medical codes.

The vector embeddings may also incorporate demographic factors and social determinants of health to provide more comprehensive and contextually appropriate recommendations. These embeddings may capture patterns related to age groups, geographic locations, socioeconomic factors, and population health trends. By including these demographic considerations in the vector space, the system may identify more relevant reference cases and generate more appropriate documentation and coding suggestions. For example, when processing a pediatric case, the embeddings may automatically prioritize age-appropriate reference records and coding guidelines, while for geriatric patients, the system may consider common comorbidities and age-specific treatment protocols.

The integration of these components within the clinical workflow platform 300 offers several advantages. It may reduce the time healthcare providers spend on documentation and coding tasks, allowing them to focus more on patient care. The automated generation of SDRs and suggested medical codes may help minimize the risk of human error and improve the consistency and completeness of medical records. Additionally, the platform's ability to learn from historical data and adapt to individual provider preferences may lead to improvements in accuracy and relevance of its outputs over time. In some cases, the platform could incorporate natural language generation capabilities to produce human-readable summaries of the structured data records.

Furthermore, the clinical workflow platform 300 may be designed with flexibility and scalability in mind. Its modular architecture may allow for updates and additions to keep pace with evolving healthcare standards and practices. For example, new coding systems or documentation requirements can be incorporated by updating the relevant databases and pipelines without requiring a complete overhaul of the system. This adaptability may help healthcare providers maintain compliance with changing regulations while benefiting from advancements in medical informatics and artificial intelligence. In some implementations, the platform may include an API layer to facilitate integration with external systems and third-party applications, further extending its capabilities and interoperability.

Each of the input record pipeline 302, the structuring pipeline 304, the service engine system 306, the SDR database 308, the code database 310, and the vector database 312 may be generically referred to as a system component. A system component refers to one or more devices and/or applications. Where a system component is or refers to a device, the system component can be, be similar to, include, or be included in a computing system, which can include one or more computing devices (e.g., one or more of the computing device 200 of FIG. 2). Where a system component is or refers to an application, the system component can be, be similar to, include, or be included in an instance of software running on a device (e.g., a computing device). In some implementations, a system component can be implemented as a single physical unit or as a combination of physical units. In some implementations, a single physical unit can include multiple components.

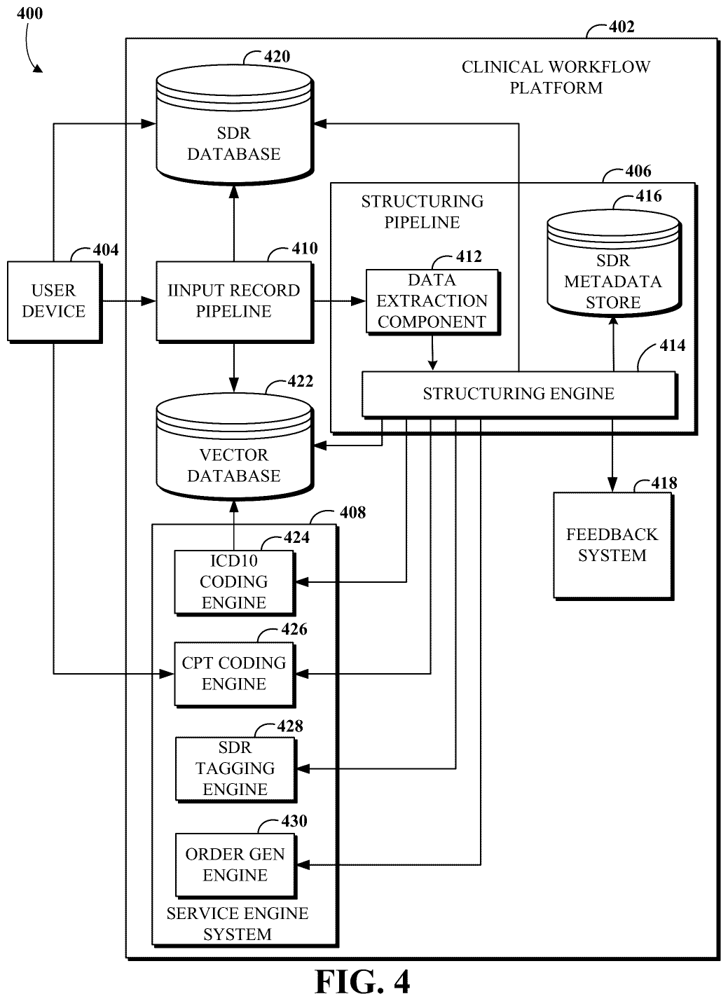

FIG. 4 is a block schematic diagram showing an example of a machine-learning-based clinical workflow system 400. As shown, the system 400 includes a clinical workflow platform 402 and a user device 404. The clinical workflow platform 402 may be, be similar to, include, or be included in the clinical workflow platform 300 shown in FIG. 300 and/or the clinical workflow platform 102 shown in FIG. 1. The user device 404 may be, be similar to, include, or be included in the user device 104 shown in FIG. 1. As shown, the clinical workflow platform 402 may include a structuring pipeline 406, a service engine system 408, and an input record pipeline 410.

The clinical workflow platform 402 functions as the central component of the system 400, incorporating multiple interconnected elements designed to process and analyze clinical data. The clinical workflow platform 402 includes a structuring pipeline 406, which may be configured to transform raw input data into a structured format. The structuring pipeline 406 utilizes advanced natural language processing techniques and machine learning algorithms to extract pertinent clinical information from input records. The structuring pipeline 406 is configured to identify components of a patient encounter, such as symptoms, diagnoses, treatments, and follow-up plans, and organize them into an SDR. The structuring pipeline 406 may employ deep learning models to enhance extraction accuracy. In some aspects, the structuring pipeline 406 may incorporate rule-based systems alongside machine learning for a hybrid approach. The structuring pipeline 406 may be designed to handle multi-modal inputs, processing textual data from clinical notes in conjunction with visual data from medical imaging or numerical data from lab results. This multi-modal capability may allow for a more comprehensive understanding of the patient's condition, potentially leading to more accurate SDRs.

The service engine system 408 may be configured to perform various retrieval-augmented machine learning operations on the structured data. The service engine system 408 may include multiple specialized engines, such as an ICD10 coding engine 424, a CPT coding engine 426, an SDR tagging engine 428, and an order generation engine 430. The ICD10 coding engine 426 may be configured to generate appropriate ICD-10 diagnostic codes based on the information contained in the SDRs. Similarly, the CPT coding engine 426 may be configured to assign CPT codes to accurately represent the procedures and services provided during the patient encounter.

The SDR tagging engine 428 may be configured to add metadata and tags to the structured data records. This tagging process enhances the searchability and categorization of the SDRs, facilitating easier retrieval and analysis of patient information. The order generation engine 430 may automate the creation of medical orders, such as prescriptions, lab tests, or imaging studies, as indicated by the clinical data contained in the SDR. This automation helps streamline the clinical workflow and reduces the potential for errors in order entry.

The input record pipeline 410 serves as the entry point for clinical data associated with patient encounters. The input record pipeline 410 is designed to handle various types of input records, including text data from written notes, audio data from recorded conversations between healthcare providers and patients, and even video data from telemedicine sessions. The input record pipeline 410 processes these diverse data types and prepares them for further analysis and structuring by the other components of the clinical workflow platform 402.

To support its operations, the clinical workflow platform 402 incorporates several databases. The SDR database 420 serves as a repository for structured data records, allowing for storage and retrieval of patient encounter information. This database stores current SDRs and maintains historical records, which can be valuable for tracking patient progress over time and identifying trends in healthcare delivery. The vector database 422 stores vector embeddings, which are numerical representations of data in a high-dimensional space that capture semantic relationships and contextual information. These vector embeddings enable sophisticated similarity searches and information retrieval operations, which facilitate the platform's retrieval-augmented machine learning capabilities.

The system 400 also includes a feedback system 418, which plays a critical role in continuously improving the accuracy and relevance of the platform's outputs. After an SDR is generated and processed by the various engines, it may be presented to a clinician through the user device 404 for review and potential editing. The feedback provided by the clinician is then processed by the feedback system 418 and used to update the machine learning models and refine the system's performance. This feedback loop allows the system to learn from each interaction and adapt to individual clinician preferences and institutional guidelines.

The data extraction component 412 within the structuring pipeline 406 is responsible for parsing the input records and identifying relevant clinical information. This component utilizes natural language processing techniques to extract key data points from unstructured text, such as patient demographics, medical history, current symptoms, and treatment plans. The extracted information is then passed to the structuring engine 414, which organizes it into a standardized SDR format.

The SDR metadata store 416 maintains information about the structure and content of the SDRs. This metadata may include details such as the types of fields present in each SDR, the data formats used, and the relationships between different data elements. The metadata store helps ensure consistency in the structure of SDRs across different patient encounters and facilitates efficient querying and analysis of the stored data.

The user device 404 serves as the interface between healthcare providers and the clinical workflow platform 402. It may be a desktop computer, laptop, tablet, or mobile device that allows clinicians to input patient encounter information, review generated SDRs and suggested codes, or provide feedback to the system. The user device 404 may run a specialized application or access the platform through a web interface, providing a user-friendly experience for healthcare providers to interact with the system.

Some implementations of the clinical workflow system 400 may be configured to reduce the time healthcare providers spend on documentation and coding tasks. By automating the generation of SDRs and suggesting appropriate ICD-10 and CPT codes, the system allows clinicians to focus more on patient care rather than administrative tasks. This not only improves the efficiency of healthcare delivery but also has the potential to enhance the quality of patient interactions by freeing up more time for direct patient care.

The system's use of retrieval-augmented machine learning techniques, facilitated by the vector database 422, allows for more accurate and contextually relevant outputs. For example, when generating an SDR or suggesting codes, the system can quickly identify and reference similar historical cases or relevant clinical guidelines. This capability may be particularly valuable in handling rare or complex cases where the base machine learning model's training data may be limited. By accessing and utilizing this external knowledge, the system can provide more informed and reliable outputs even for uncommon medical scenarios.

In some implementations, the system 400 may operate in real-time during live patient encounters, processing the conversation between clinician and patient as it occurs. The input record pipeline 410 may receive audio input through a microphone on the user device 404 or other recording device, converting the spoken dialogue into text while maintaining speaker attribution and temporal sequence. As the conversation progresses, the system 400 may analyze the content using natural language processing to identify clinically relevant information, medical terminology, symptoms, and potential diagnoses. This real-time processing may allow the system 400 to provide immediate feedback and suggestions through the user device 404, which may be configured to display prompts discretely without disrupting the natural flow of the patient encounter.

The system 400 may leverage its understanding of medical coding requirements and documentation standards to provide contextual prompts to the clinician during the encounter. For example, if a patient mentions symptoms that could qualify for a higher complexity E&M code, the system 400 may suggest follow-up questions through the user interface to gather additional information needed to support that code level. The system 400 may display these suggestions unobtrusively, such as through a small notification or subtle highlight on the clinician's screen. In some cases, the system 400 may recognize when certain elements of the patient history or examination would be relevant for specific diagnostic codes and may prompt the clinician to explore those areas further. For instance, if a patient's symptoms suggest a particular condition, the system 400 may recommend additional assessment questions that would help differentiate between similar diagnoses or establish the severity level needed for accurate coding. The system 400 may also track the documentation requirements for different code levels in real-time, alerting the clinician when additional information may be needed to support optimal code selection while the patient is still present.

Each of the clinical workflow platform 402, the user device 404, structuring pipeline 406, the service engine system 408, the input record pipeline 410, the data extraction component 412, the structuring engine 414, the SDR metadata store 416, the feedback system 418, the SDR database 420, the vector database 422, the ICD10 coding engine 424, the CPT coding engine 426, SDR tagging engine 428, and the order generation engine 430 may be generically referred to as a system component. A system component refers to one or more devices and/or applications. Where a system component is or refers to a device, the system component can be, be similar to, include, or be included in a computing system, which can include one or more computing devices (e.g., one or more of the computing device 200 of FIG. 2). Where a system component is or refers to an application, the system component can be, be similar to, include, or be included in an instance of software running on a device (e.g., a computing device). In some implementations, a system component can be implemented as a single physical unit or as a combination of physical units. In some implementations, a single physical unit can include multiple components.

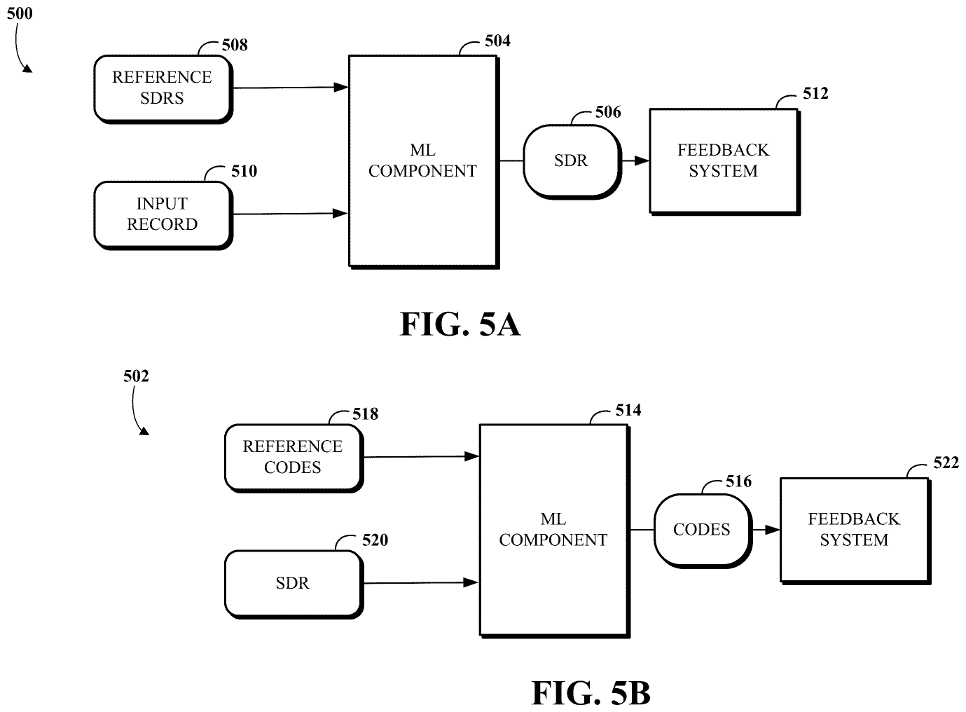

FIGS. 5A and 5B are schematic diagrams illustrating example processes associated with a machine-learning-based clinical workflow. The processes illustrated in FIGS. 5A and 5B, as well as other processes described herein, may be implemented by any of the systems or components described above. For example, the processes may be executed by the clinical workflow platform 102 shown in FIG. 1, the computing device 200 shown in FIG. 2, the clinical workflow platform 300 shown in FIG. 3, or the clinical workflow system 400 shown in FIG. 4.

FIG. 5A shows a process 500 for generating an SDR associated with a patient encounter. The process 500 includes several components that work together to transform raw clinical data into a structured format suitable for further analysis and use in healthcare settings.

The process 500 begins with an input record 510, which may include clinical data associated with a patient encounter. This input record 510 may take various forms, including but not limited to text data from written notes, audio data from recorded conversations between healthcare providers and patients, or video data from telemedicine sessions. In some implementations, the input record 510 may also include structured data entered directly into electronic health record (EHR) systems, data from wearable devices, or remote patient monitoring systems.

The input record 510 is provided as input to an ML component 504. The ML component 504 may be configured to perform a RAG ML operation. This ML component 504 may be implemented using various machine learning models or algorithms, such as neural networks, decision trees, or ensemble methods. In some implementations, the ML component 504 may utilize deep learning techniques to improve its ability to extract relevant information from the input record 510.

The ML component 504 also receives, as input, reference SDRs 508. These reference SDRs 508 serve as a knowledge base for the ML component 504, providing examples of well-structured clinical data that can guide the generation of new SDRs. The reference SDRs 508 may be stored in a database and may include historical patient records, curated examples of best practices in clinical documentation, or standardized templates for specific types of patient encounters. In some implementations, the reference SDRs 508 may be continuously updated with new, high-quality examples to improve the performance of the ML component 504 over time.

Using the input record 510 and the reference SDRs 508, the ML component 504 generates an SDR 506. The SDR 506 is a structured representation of the clinical data contained in the input record 510. This structured format may include various sections such as patient demographics, chief complaint, medical history, physical examination findings, assessment, and treatment plan. In some implementations, the SDR 506 may follow a standardized format such as the SOAP (Subjective, Objective, Assessment, Plan) note structure commonly used in healthcare settings.

The generated SDR 506 is then passed to a feedback system 512. The feedback system 512 may be configured for improving the accuracy and relevance of the generated SDRs over time. It may allow healthcare providers to review and edit the generated SDR 506, providing valuable input on the structure, content, and accuracy of the record. In some implementations, the feedback system 512 may incorporate machine learning techniques to automatically identify patterns in user edits and use this information to improve the performance of the ML component 504.

FIG. 5B illustrates a related process 502 for generating diagnostic codes based on the structured data record. This process demonstrates how the system can leverage the structured information created in process 500 to assist with medical coding tasks.

The process 502 begins with an SDR 520, which may be the same as or derived from the SDR 506 generated in process 500. This SDR 520 serves as the basis for generating appropriate diagnostic codes for the patient encounter.

The SDR 520 is provided as input to an ML component 514. Similar to the ML component 504 in process 500, this ML component 514 is configured to perform a RAG ML operation. However, in this case, the ML component 514 is specifically trained to analyze structured clinical data and generate relevant diagnostic codes.

The ML component 514 also receives input from reference codes 518. These reference codes 518 may include comprehensive databases of diagnostic codes, such as ICD-10 codes, along with their descriptions, usage guidelines, and exclusion criteria. In some implementations, the reference codes 518 may also include historical data on code usage patterns, which can help the ML component 514 identify the most appropriate codes for a given clinical scenario.

Using the SDR 520 and the reference codes 518, the ML component 514 generates a set of codes 516. These codes 516 may include primary and secondary diagnostic codes, as well as procedure codes if applicable. The codes 516 are designed to accurately represent the clinical information contained in the SDR 520 in a standardized, codified format suitable for billing, research, and other administrative purposes.

The generated codes 516 are then passed to a feedback system 522. Similar to the feedback system 512 in process 500, this feedback system 522 allows for human review and correction of the generated codes. In a typical implementation, experienced medical coders or healthcare providers may review the suggested codes, make any necessary adjustments, and provide feedback to the system. This feedback can be used to continuously improve the accuracy of the ML component 514.

The processes illustrated in FIGS. 5A and 5B demonstrate a comprehensive approach to clinical documentation and coding using machine learning techniques. By leveraging historical data, standardized reference materials, and human expertise, these processes aim to improve the efficiency and accuracy of clinical workflows. The use of structured data records and standardized coding systems facilitates better data interoperability, more accurate billing practices, and improved opportunities for clinical research and quality improvement initiatives.

In some implementations, the processes 500 and 502 may be combined into a single, end-to-end process that takes raw clinical data as input and produces both structured documentation and diagnostic codes as output. This integrated approach could potentially streamline workflows even further and ensure greater consistency between the documentation and coding processes. Some implementations may involve the use of multiple ML components working in parallel, each specialized for different aspects of the clinical documentation or coding tasks. For example, one ML component might focus on extracting patient history information, while another specializes in interpreting physical examination findings. The outputs from these specialized components could then be combined to create the final SDR or code set. In some implementations, the feedback systems 512 and 522 may be implemented with natural language processing capabilities to automatically interpret and categorize user feedback, making it easier to incorporate this information into the ongoing training of the ML components. This could potentially accelerate the learning process and allow the system to adapt more quickly to new clinical scenarios or coding guidelines.

Each of the ML component 504, the feedback system 512, the ML component 514, and the feedback system 522 may be generically referred to as a system component. A system component refers to one or more devices and/or applications. Where a system component is or refers to a device, the system component can be, be similar to, include, or be included in a computing system, which can include one or more computing devices (e.g., one or more of the computing device 200 of FIG. 2). Where a system component is or refers to an application, the system component can be, be similar to, include, or be included in an instance of software running on a device (e.g., a computing device). In some implementations, a system component can be implemented as a single physical unit or as a combination of physical units. In some implementations, a single physical unit can include multiple components.

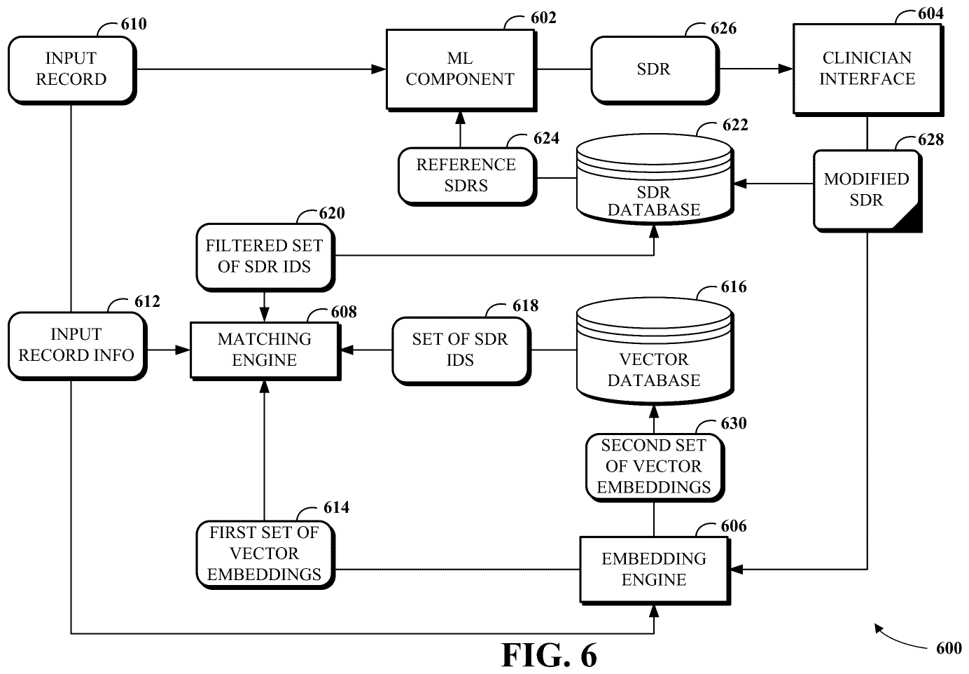

FIG. 6 is a schematic diagram illustrating another example process 600 associated with a machine-learning-based clinical workflow. As shown, the process 600 may be implemented using an ML component 602, a clinician interface 604, an embedding engine 606, a matching engine 608, and an SDR database 622. Any one or more of the ML component 602, the clinician interface 604, the embedding engine 606, the matching engine 608, and the SDR database 622 may be, be similar to, include, or be included in the system 400 shown in FIG. 4 (or corresponding components thereof), the clinical workflow platform 300 shown in FIG. 3 (or corresponding components thereof), the computing device 200 shown in FIG. 2 (or corresponding components thereof), or the system 100 shown in FIG. 1 (or corresponding components thereof).

The process 600 may include obtaining an input record 610 that may include clinical data associated with a patient encounter. In some implementations, the input record 610 may include a transcript, which may contain at least one of text data, audio data, or video data related to the patient encounter.

The process 600 may include providing the input record 610 as input to an ML component 602 configured to perform a RAG ML operation. The ML component 602 may be implemented using various machine learning models or algorithms, such as large language models (LLMs), neural networks, decision trees, or ensemble methods. In some implementations, the ML component 602 may utilize deep learning techniques to improve its ability to extract relevant information from the input record.

The process 600 may include generating a set of vector embeddings corresponding to the input record. This may be performed by an embedding engine 606, which may create a first set of vector embeddings 614 based on the input record 610 and input record info 612. The input record info 612 may include additional metadata or contextual information about the patient encounter, such as the patient's medical history, demographic information, or the healthcare provider's specialty.

The process 600 may include identifying, using a vector similarity operation associated with the set of vector embeddings and a vector database 616 that includes at least one additional set of vector embeddings, a set of reference records for use in the RAG ML operation. This identification may be performed by a matching engine 608, which may search the vector database 616 to find a set of SDR IDs 618 that are most similar to the first set of vector embeddings 614.

Vector similarity operations in this context may include various techniques for comparing and measuring the similarity between vector embeddings. These operations can be used to identify relevant reference records or similar clinical cases based on the vector representations of input data. Some examples of vector similarity operations that may be used in this context include cosine similarity, which measures the cosine of the angle between two vectors. This operation may be particularly useful for comparing high-dimensional vector embeddings, as it focuses on the orientation of the vectors rather than their magnitude. Vector similarity operations may include Euclidean distance, which calculates the straight-line distance between two points in vector space. This operation may be used to find the nearest neighbors of a given vector embedding. Vector similarity operations may include dot product, which computes the sum of the products of corresponding elements in two vectors. This operation may be used as a simple measure of similarity between vector embeddings. Vector similarity operations may include Jaccard similarity, which measures the overlap between sets represented by binary vectors. This operation may be useful for comparing vector embeddings that represent the presence or absence of specific features in clinical data. Vector similarity operations may include Manhattan distance, also known as L1 distance or city block distance, which calculates the sum of the absolute differences between vector elements. This operation may be used as an alternative to Euclidean distance in certain scenarios. In some implementations, the system may use a combination of these similarity operations or apply different operations depending on the specific characteristics of the vector embeddings being compared.

In some implementations, the process 600 may include determining a set of structured data record metadata associated with the input record. In some implementations, SDR IDs may be similar to summarize job names. These identifiers may serve as unique labels for structured data records within the system. For example, an SDR ID might be “ORTHO_KNEE_001” for an orthopedic note related to a knee examination, or “CARDIO_ECHO_002” for a cardiology note involving an echocardiogram. Summarize job names may follow a similar naming convention, potentially including additional metadata. For instance, a summarize job name could be “SUMMARIZE_ORTHO_KNEE_001_DR_SMITH” or “SUMMARIZE CARDIO_ECHO_002_20230515”. These names may include information such as the specialty, procedure type, a unique identifier, the physician's name, or the date of the encounter. In some cases, the system may use these identifiers interchangeably or map between them. For example, when searching the vector database, the system may use an SDR ID to retrieve the corresponding summarize job name, or vice versa. This mapping may allow for flexible querying and retrieval of relevant information across different components of the system.

The matching engine 608 may use metadata to refine its search and produce a filtered set of SDR IDs 620. This filtering process may involve applying a context filter to ensure that the identified reference records are relevant to the specific patient encounter. In some implementations, the matching engine 608 may employ a technique of identifying m similar names and selecting the top k matches. This approach may help in retrieving the most relevant reference records for the current patient encounter. For example, the matching engine 608 may first identify m SDR IDs or summarize job names that are most similar to the current input record based on vector similarity operations. The value of m may be predetermined or dynamically adjusted based on factors such as the specificity of the input record or the desired level of granularity in the matching process.

Once the m similar names are identified, the matching engine 608 may then select the top k matches from this set. The selection of the top k matches may be based on additional criteria such as recency of the records, relevance scores, specific attributes of the patient encounter such as relevant body part or medical specialty. The value of k may be smaller than m, allowing for a more focused set of reference records to be used in the subsequent steps of the RAG ML operation. This two-step process of first identifying m similar names and then selecting the top k matches may provide a balance between casting a wide net for potential matches and focusing on the most relevant records. It may help in handling cases where there are many similar records but only a subset are highly relevant to the current patient encounter.

In some implementations, the values of m and k may be adjustable parameters that can be fine-tuned based on the performance of the system or specific requirements of different clinical scenarios. For instance, a larger m value may be used for rare or complex cases to ensure a broader range of potential matches is considered, while a smaller k value may be used for more routine encounters to focus on the most directly relevant reference records. The matching engine 608 may also incorporate feedback from the clinician interface 604 to refine its selection process over time. For example, if certain types of reference records are consistently found to be more useful by clinicians, the matching engine 608 may adjust its selection criteria to prioritize similar records in future searches.

The matching engine 608 may retrieve the reference records based on the identified SDR IDs through a multi-step process that leverages the vector database and additional filtering techniques. After identifying the set of SDR IDs, the matching engine 608 may query the SDR database to fetch the corresponding reference records. In some implementations, the matching engine 608 may use the SDR IDs as keys to directly access the reference records stored in the SDR database. The SDR database may be structured to allow efficient retrieval of records based on their unique identifiers. The matching engine 608 may also employ caching mechanisms to improve retrieval speed for frequently accessed reference records. This approach may involve maintaining a local cache of recently or commonly used reference records, allowing for faster access without the need to query the main database for every request. In some cases, the matching engine 608 may implement a batched retrieval process, where multiple SDR IDs are grouped together and retrieved in a single database query. This method may help optimize database access and reduce overall retrieval time, especially when dealing with a large number of reference records.

In some implementations, the matching engine 608 may also apply additional filters or transformations to the retrieved reference records before passing them to the ML component. This post-retrieval processing may include, for example, formatting the reference records to ensure compatibility with the ML component's input requirements, extracting specific sections or fields from the reference records that are most relevant to the current patient encounter, or applying privacy filters to remove or mask sensitive patient information not necessary for the ML operation, among other examples. In some implementations, the matching engine 608 may employ a hierarchical retrieval strategy. It may first fetch a subset of the reference record, such as metadata or summary information, and then retrieve the full record only if needed based on initial analysis or ML component requirements.

The matching engine 608 may also incorporate versioning in its retrieval process. If multiple versions of a reference record exist, the matching engine 608 may select the most appropriate version based on factors such as the date of the current patient encounter or specific version tags associated with the SDR IDs. In cases where the SDR database is distributed across multiple storage systems or locations, the matching engine 608 may implement a federated query approach. This method allows the matching engine 608 to retrieve reference records from various data sources while presenting a unified interface to the ML component.

The process 600 may include generating, using the ML component 602 and based on the input record and the set of reference records, an SDR 626 associated with the patient encounter. The ML component 602 may retrieve reference SDRs 624 from an SDR database 622 based on the filtered set of SDR IDs 620. These reference SDRs 624 may serve as examples or templates to guide the generation of the new SDR 626.

In some implementations, the ML component 602 may be an RAG LLM that may generate the SDR through a multi-step process that leverages both the input record and the retrieved reference records. The ML component may first analyze the input record, which may include the transcript of the patient encounter and any associated metadata, to extract key clinical information. This analysis may involve natural language processing techniques to identify relevant medical terms, symptoms, diagnoses, and treatment plans mentioned in the input record.

The ML component may then examine the set of reference records. These reference records may serve as examples of well-structured clinical documentation for similar patient encounters. The RAG LLM may compare the extracted information from the input record with the content and structure of the reference records to identify patterns and best practices in organizing clinical data. Using this combined information, the RAG LLM may begin to construct the new SDR. The LLM may generate sections of the SDR sequentially, potentially following a predefined template or structure commonly used in clinical documentation. For each section, the LLM may draw upon relevant information from the input record while referencing the structure and content of similar sections in the reference records.

In some cases, the RAG LLM may employ a technique called “few-shot learning” where it uses the reference records as examples to guide its generation process. This approach may allow the LLM to adapt its output to match the style and format of existing records in the system, even if it hasn't been explicitly trained on that specific format. The ML component may also incorporate domain-specific knowledge encoded in its training data to ensure the generated SDR adheres to medical best practices and terminology. This may include automatically expanding abbreviations, correcting common misspellings of medical terms, or inferring implied information based on the context of the patient encounter.

As the RAG LLM generates each section of the SDR, it may perform self-consistency checks to ensure the information is coherent and logically structured. This may involve cross-referencing different sections of the SDR to avoid contradictions and ensure all relevant information from the input record is appropriately captured. In some implementations, the ML component may assign confidence scores to different parts of the generated SDR. These scores may reflect the LLM's certainty about the accuracy or appropriateness of the generated content. Sections with lower confidence scores may be flagged for human review or may trigger the LLM to seek additional information from the reference records or other knowledge sources.

The RAG LLM may also generate metadata for the SDR, such as tags or categories that describe the content of the record. These metadata elements may be based on both the content of the generated SDR and patterns observed in the metadata of the reference records. In some aspects, the ML component may implement an iterative refinement process, where it generates an initial draft of the SDR and then makes multiple passes to improve and refine the content. Each iteration may focus on different aspects of the SDR, such as completeness, consistency, adherence to formatting standards, or use of appropriate medical terminology.

The process 600 may include outputting structured record information, corresponding to the structured data record 626, to cause a display of a user device to present a representation of the SDR 626. This may be accomplished through a clinician interface 604, which may allow healthcare providers to review and interact with the generated SDR 626. In some implementations, the process 600 may include providing the SDR 626 to the clinician interface 604 for review and potential editing. The clinician interface 604 may receive clinician feedback associated with the SDR 626, which may be used to generate a modified SDR 628. This feedback mechanism allows for continuous improvement of the system's performance and adaptation to individual clinician preferences.

The process 600 may include outputting the SDR 626 or the modified SDR 628 for use by an ML-based service engine system in performing one or more retrieval-augmented machine learning operations. These operations may include a diagnosis classification code prediction operation, a procedure classification code prediction operation, or an order generation operation, among others. In some implementations, the process 600 may include generating, based on the clinician feedback, a second set of vector embeddings 630 corresponding to the modified SDR 628. The embedding engine 606 may create these new vector embeddings, which may then be stored in the vector database 616 for future use. This allows the system to continuously learn and improve its performance based on clinician input. The process 600 may include providing the SDR 626 or the modified SDR 628 for output to an electronic health record. This integration with existing healthcare information systems ensures that the generated structured data can be easily incorporated into established clinical workflows and documentation processes.

Some implementations of the process 600 may include additional steps or variations of the described steps. For example, the ML component 602 may employ multiple specialized sub-models, each focused on extracting specific types of clinical information. The vector database 616 may be implemented using various database technologies, such as graph databases or distributed NoSQL systems, to optimize performance and scalability. The clinician interface 604 may incorporate natural language processing capabilities to allow for voice-based interactions or free-text editing of the generated SDRs. These and other variations may be implemented to adapt the process 600 to specific clinical settings or requirements.

In some implementations, the system may incorporate several features designed to optimize revenue capture and prevent revenue leakage throughout the clinical documentation and coding process. In some implementations, the system may analyze historical claims data to identify patterns of denied claims or downcoding, using this information to proactively suggest documentation improvements that may help prevent similar issues. The system may maintain a database of payer-specific requirements and coding guidelines, which may be used to customize documentation suggestions based on the specific insurance plans associated with each patient encounter.

In some aspects, the system may implement real-time validation checks that compare selected codes against documentation requirements, alerting clinicians when additional documentation may be needed to support specific code levels. The system may also track commonly missed documentation elements or coding opportunities across different specialties and practice settings, using this information to generate targeted suggestions for improving documentation completeness.

In some cases, the system may implement automated auditing capabilities that regularly review documentation and coding patterns to identify potential revenue leakage. These audits may examine factors such as the frequency of certain code combinations, the distribution of evaluation and management code levels, and the completeness of supporting documentation. The system may generate reports highlighting opportunities for improvement and suggesting specific actions to address identified issues.

Each of the ML component 602, the clinician interface 604, the embedding engine 606, the matching engine 608, and the SDR database 622 may be generically referred to as a system component. A system component refers to one or more devices and/or applications. Where a system component is or refers to a device, the system component can be, be similar to, include, or be included in a computing system, which can include one or more computing devices (e.g., one or more of the computing device 200 of FIG. 2). Where a system component is or refers to an application, the system component can be, be similar to, include, or be included in an instance of software running on a device (e.g., a computing device). In some implementations, a system component can be implemented as a single physical unit or as a combination of physical units. In some implementations, a single physical unit can include multiple components.

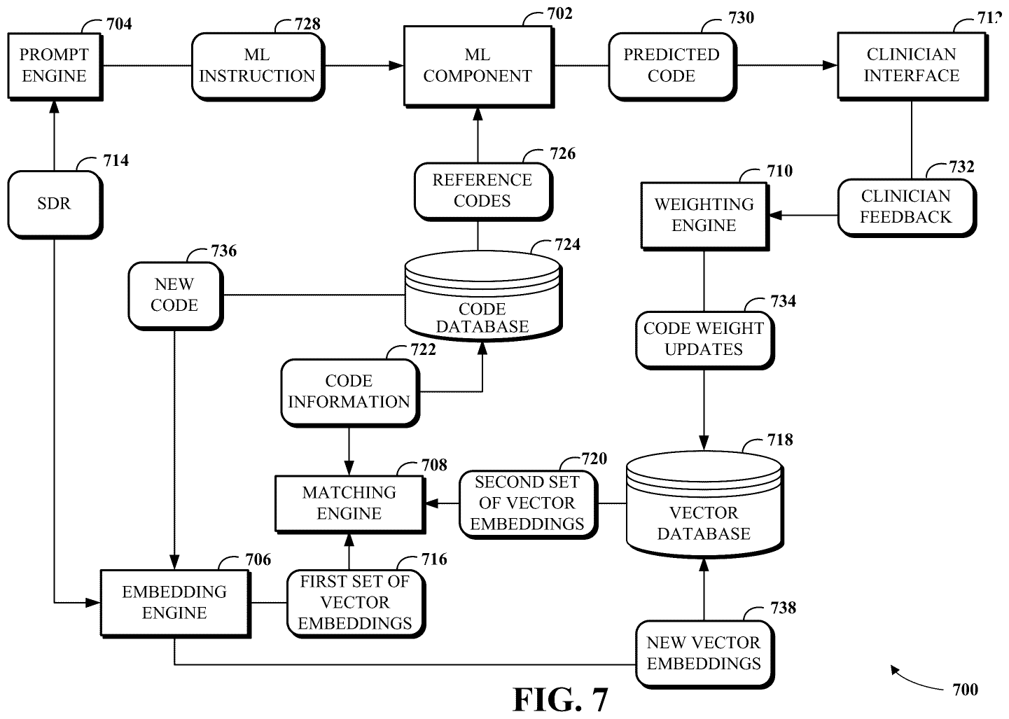

FIG. 7 is a schematic diagram illustrating another example process associated with a machine-learning-based clinical workflow.

As shown, the process 700 may be implemented using an ML component 702, a prompt engine 704, an embedding engine 706, a matching engine 708, a weighting engine 710, a clinician interface 712, a vector database 718, and a code database 724 Any one or more of the ML component 702, the prompt engine 704, the embedding engine 706, the matching engine 708, the weighting engine 710, the clinician interface 712, the vector database 718, and the code database 724 may be, be similar to, include, or be included in the system 400 shown in FIG. 4 (or corresponding components thereof), the clinical workflow platform 300 shown in FIG. 3 (or corresponding components thereof), the computing device 200 shown in FIG. 2 (or corresponding components thereof), or the system 100 shown in FIG. 1 (or corresponding components thereof).

The process 700 may include generating a machine learning instruction based on an SDR 714 associated with a patient encounter. The SDR 714 may include clinical information such as patient demographics, medical history, symptoms, examination findings, diagnoses, and treatment plans. In some implementations, the SDR 714 may be generated from an input record comprising clinical data associated with the patient encounter, such as text data from written notes, audio data from recorded conversations, or video data from telemedicine sessions.

The process 700 may include providing the machine learning instruction to an ML component 702 configured to perform a RAG ML operation. The ML component 702 may be implemented using various machine learning models or algorithms, such as large language models, neural networks, or ensemble methods. In some implementations, the ML component 702 may utilize deep learning techniques to improve its ability to process and analyze clinical data.

The process 700 may include obtaining a set of vector embeddings corresponding to the SDR 714. An embedding engine 706 may be responsible for generating a first set of vector embeddings 716 based on the SDR 714. Vector embeddings are numerical representations of data in a high-dimensional space that capture semantic relationships and contextual information. In some implementations, the embedding engine 706 may use techniques such as word2vec, BERT, or other transformer-based models to create these embeddings.

The process 700 may include identifying, using a vector similarity operation associated with the set of vector embeddings and a vector database 718 that includes at least one additional set of vector embeddings, a set of reference codes 726 for use in the RAG ML operation. A matching engine 708 may perform this identification by searching the vector database 718 to find a second set of vector embeddings 720 that are most similar to the first set of vector embeddings 716. The vector similarity operation may include techniques such as cosine similarity, Euclidean distance, or dot product calculations.

The vector database 718 may contain sets of embedding vectors associated with various medical coding systems, electronic health record systems, code descriptions, medical guidelines, code exclusions, synonyms, or disease names. This comprehensive database allows for efficient retrieval of relevant reference information based on the context of the current patient encounter. In some implementations, the vector database 718 may be regularly updated to reflect changes in medical coding standards and practices.

A code database 724 may store detailed information about medical codes, including ICD-10 codes, CPT codes, and healthcare common procedure coding system (HCPCS) codes. The code database 724 may provide code information 722 to the matching engine 708, which may be used in conjunction with the vector embeddings to identify the most appropriate reference codes 726.

A prompt engine 704 may be used to generate a machine learning instruction 728 based on the SDR 714. The prompt engine 704 may be configured to extract relevant information from the SDR 714 and formulate appropriate instructions for the ML component 702. In some implementations, the prompt engine 704 may use natural language processing techniques to interpret the content of the SDR 714 and generate context-specific instructions.

The prompt engine 704 may employ various techniques to generate the machine learning instruction 728 based on the SDR 714. In some implementations, the prompt engine 704 may use a template-based approach, where predefined templates are filled with specific information extracted from the SDR 714. These templates may be designed to capture different aspects of the clinical encounter, such as chief complaint, medical history, or physical examination findings. The prompt engine 704 may utilize natural language processing (NLP) techniques to analyze the content of the SDR 714 and identify key clinical concepts, relationships, and context. This analysis may involve named entity recognition to identify medical terms, symptom extraction, and semantic role labeling to understand the relationships between different elements of the clinical narrative.

In some cases, the prompt engine 704 may incorporate a hierarchical approach to generate the machine learning instruction 728. This may involve first creating a high-level summary of the patient encounter, followed by more detailed instructions for specific sections of the SDR 714. The prompt engine 704 may also consider the intended use of the machine learning instruction, such as generating diagnostic codes or treatment recommendations, to tailor the content and structure of the instruction accordingly. The prompt engine 704 may leverage domain-specific knowledge bases or ontologies to enhance the relevance and accuracy of the generated instruction. This may include mapping clinical terms to standardized medical vocabularies, such as SNOMED CT or RxNorm, to ensure consistency and facilitate interoperability with other healthcare systems.

In some implementations, the prompt engine 704 may incorporate a dynamic prompting strategy, where the content and structure of the machine learning instruction 728 are adjusted based on the complexity of the patient case, the specialty of the healthcare provider, or the specific requirements of the healthcare organization. This adaptability may allow for more targeted and efficient processing by the ML component 702. The prompt engine 704 may include mechanisms for handling uncertainty or incomplete information in the SDR 714. This may involve generating multiple alternative instructions or including specific queries within the instruction to prompt the ML component 702 to seek additional information or clarification.

The process 700 may include generating, using the ML component 702 and based on the machine learning instruction 728 and the set of reference codes, a predicted diagnosis classification code 730. The predicted diagnosis classification code 730 may include primary diagnosis codes, primary procedure codes, or other relevant medical codes. In some implementations, the predicted diagnosis classification code 730 may be based on billing codes, diagnostic codes, procedural codes, or facility information extracted from the SDR 714 and the reference codes.

The ML component 702 may generate the predicted diagnosis classification code 730 through a multi-step process that leverages both the machine learning instruction 728 and the set of reference codes. Initially, the ML component 702 may analyze the machine learning instruction 728, which may contain key information extracted from the SDR 714. This analysis may involve natural language processing techniques to identify relevant medical terms, symptoms, diagnoses, and treatment plans mentioned in the instruction.

The ML component 702 may then examine the set of reference codes 726 provided by the matching engine 708. These reference codes 726 may serve as examples of appropriate coding for similar patient encounters. The ML component 702 may compare the information extracted from the machine learning instruction 728 with the content and context of the reference codes to identify patterns and best practices in medical coding. Using this combined information, the ML component 702 may begin to construct potential diagnosis classification codes. The component may generate multiple code candidates, each with an associated confidence score based on the similarity between the current patient encounter and the reference cases.

In some implementations, the ML component 702 may employ a technique called “few-shot learning” where it uses the reference codes 726 as examples to guide its code generation process. This approach may allow the ML component to adapt its output to match the coding practices specific to the healthcare organization or specialty, even if it hasn't been explicitly trained on that specific coding style. The ML component 702 may also incorporate domain-specific knowledge encoded in its training data to ensure the generated codes adhere to current coding guidelines and best practices. This may include automatically expanding abbreviations, interpreting clinical terminology, or inferring implied diagnoses based on the context of the patient encounter.

As the ML component 702 generates potential diagnosis classification codes, it may perform self-consistency checks to ensure the codes are logically coherent and clinically appropriate. This may involve cross-referencing different sections of the SDR 714 to avoid contradictions and ensure all relevant information is appropriately captured in the coding. In some cases, the ML component 702 may assign confidence scores to different aspects of the generated codes. These scores may reflect the component's certainty about the accuracy or appropriateness of the code assignments. Codes with lower confidence scores may be flagged for human review or may trigger the ML component to seek additional information from the reference codes or other knowledge sources.

The process 700 may include providing an output configured to cause a display of a user device to present a representation of the predicted diagnosis classification code 730. This output may be provided to a clinician interface 712, allowing healthcare providers to review and interact with the generated codes. The clinician interface 712 may be implemented as a web-based application, a mobile app, or integrated into existing electronic health record systems.

The process 700 may include receiving, from the user device or clinician interface 712, clinician feedback 732 associated with the predicted diagnosis classification code 730. This feedback mechanism allows for continuous improvement of the system's performance and adaptation to individual clinician preferences or institutional guidelines. In some implementations, the clinician feedback 732 may be used to update the ML component 702 or refine the code selection process.

A weighting engine 710 may be used to process the clinician feedback 732 and generate code weight updates 734. These updates may be applied to the vector database 718, adjusting the importance or relevance of certain codes (e.g., the reference codes 726) based on clinician feedback 732. This adaptive approach allows the system to learn from each interaction and improve its accuracy over time.

The ML component 702 may be configured to provide detailed information about its decision-making process to the feedback system (e.g., the weighting engine 710). This information may include which reference codes 726 were most influential in generating the predicted diagnosis classification code 730, as well as the degree to which each reference code 726 contributed to the final prediction. In some implementations, the ML component 702 may assign importance scores or weights to each reference code 726 used in the prediction process. These scores may reflect the relevance and impact of each reference code 726 on the final output. The ML component 702 may then pass these scores, along with the corresponding reference codes 726, to the feedback system.

The feedback system may use this information to enhance the quality and specificity of the feedback provided to clinicians through the clinician interface 712. For example, when presenting the predicted diagnosis classification code 730 to a clinician for review, the system may highlight which reference codes 726 were most influential in generating the prediction. This may allow clinicians to better understand the reasoning behind the ML component's decision and provide more targeted feedback. Additionally, the feedback system may use the importance scores to prioritize which aspects of the prediction to focus on when soliciting clinician feedback. Reference codes 726 with higher importance scores may be given more prominence in the feedback interface, potentially leading to more valuable and impactful feedback from clinicians.

The detailed information provided by the ML component 702 may also be used to improve the system's learning process. By understanding which reference codes 726 were most influential in successful predictions (as validated by clinician feedback), the system may adjust its weighting and selection criteria for future predictions. This may lead to more accurate and relevant reference code 726 selection over time. In some cases, the ML component 702 may provide explanations or rationales for its use of specific reference codes 726. These explanations may be presented to clinicians through the feedback system, potentially increasing transparency and trust in the ML component's decision-making process. This increased transparency may encourage more thoughtful and detailed feedback from clinicians, further enhancing the system's ability to learn and improve.

The feedback system may also use the information provided by the ML component 702 to identify patterns or trends in code usage and relevance. For example, if certain reference codes 726 consistently receive high importance scores across multiple predictions, this may indicate that these codes are particularly valuable for the specific healthcare context or specialty. This information may be used to refine the content of the code database 724 or adjust the vector embeddings in the vector database 718. By facilitating this detailed exchange of information between the ML component 702 and the feedback system, the process 700 may create a more robust and adaptive learning loop. This may enable the ML component 702 to continuously refine its performance based on not just the final outcomes of its predictions, but also on the specific reasoning and reference materials used to reach those predictions.

In some implementations, the process 700 may include adding new codes 736 to the code database 724 based on clinician feedback or updates to medical coding standards. The embedding engine 706 may generate new vector embeddings 738 for these new codes, which are then added to the vector database 718. This ensures that the system remains up-to-date with the latest coding practices and can adapt to evolving healthcare standards.

The process 700 demonstrates a comprehensive approach to medical coding using machine learning techniques. By leveraging vector embeddings, retrieval-augmented generation, and continuous feedback, the system aims to improve the efficiency and accuracy of diagnosis classification in clinical workflows. The integration of multiple databases and specialized engines allows for a nuanced understanding of medical terminology and coding practices, potentially reducing errors and improving the quality of healthcare documentation.

Each of the ML component 702, the prompt engine 704, the embedding engine 706, the matching engine 708, the weighting engine 710, the clinician interface 712, the vector database 718, and the code database 724 may be generically referred to as a system component. A system component refers to one or more devices and/or applications. Where a system component is or refers to a device, the system component can be, be similar to, include, or be included in a computing system, which can include one or more computing devices (e.g., one or more of the computing device 200 of FIG. 2). Where a system component is or refers to an application, the system component can be, be similar to, include, or be included in an instance of software running on a device (e.g., a computing device). In some implementations, a system component can be implemented as a single physical unit or as a combination of physical units. In some implementations, a single physical unit can include multiple components.

To further describe some implementations in greater detail, reference is next made to examples of techniques which may be performed by or using a system for machine-learning based clinical workflows. FIG. 8 is a flowchart of an example of a technique 800 associated with a machine-learning-based clinical workflow. The technique 800 can be executed using computing devices, such as the systems, hardware, and software described with respect to FIGS. 1-7. The technique 800 can be performed, for example, by executing a machine-readable program or other computer-executable instructions, such as routines, instructions, programs, or other code. The steps, or operations, of the technique 800, or another technique, method, process, or algorithm described in connection with the implementations disclosed herein can be implemented directly in hardware, firmware, software executed by hardware, circuitry, or a combination thereof.

For simplicity of explanation, the technique 800 is depicted and described herein as a series of steps or operations. However, the steps or operations of the technique 800 can occur in various orders and/or concurrently. Additionally, other steps or operations not presented and described herein may be used. Furthermore, not all illustrated steps or operations may be required to implement a technique in accordance with the disclosed subject matter.

At 802, the technique 800 includes obtaining an input record comprising clinical data associated with a patient encounter. In some implementations, the input record includes at least one of text data, audio data, or video data. At 804, the technique 800 includes providing the input record as input to an ML component configured to perform a RAG ML operation.

At 806, the technique 800 includes generating a set of vector embeddings corresponding to the input record. At 808, the technique 800 includes identifying, using a vector similarity operation associated with the set of vector embeddings and a vector database that includes at least one additional set of vector embeddings, a set of reference records, corresponding to the at least one additional set of vector embeddings, for use in the RAG ML operation.

In some implementations, the technique 800 further includes determining a set of structured data record metadata associated with the input record, where identifying the set of reference records includes identifying at least one reference record based on the set of structured data record metadata. In some implementations, identifying the set of reference records includes searching, based on a set of structured data record metadata associated with the input record, a vector database to identify a set of structured data record identifiers, and determining, based on a context filter, a subset of the set of structured data record identifiers, where the subset of the set of structured data record identifiers corresponds to the set of reference records.

At 810, the technique 800 includes generating, using the machine learning component and based on the input record and the set of reference records, a structured data record associated with the patient encounter. At 812, the technique 800 includes outputting structured record information, corresponding to the structured data record to cause a display of a user device to present a representation of the structured data record.

In some implementations, the technique 800 includes outputting the structured data record for use by an ML-based service engine system in performing one or more retrieval-augmented machine learning operations. In some implementations, the one or more retrieval-augmented machine learning operations includes a diagnosis classification code prediction operation. In some implementations, the one or more retrieval-augmented machine learning operations includes a procedure classification code prediction operation. In some implementations, the one or more retrieval-augmented machine learning operations includes an order generation operation.

In some implementations, the technique 800 includes providing the structured data record to a clinician interface; receiving, via the clinician interface, clinician feedback associated with the structured data record; generating, based on the clinician feedback, a modified structured data record; and outputting the modified structured data record for use by an ML-based service engine system in performing one or more retrieval-augmented machine learning operations. In some implementations, the technique 800 includes providing the structured data record for output to an electronic health record.

FIG. 9 is a flowchart of an example of a technique 900 associated with a machine-learning-based clinical workflow. The technique 900 can be executed using computing devices, such as the systems, hardware, and software described with respect to FIGS. 1-7. The technique 900 can be performed, for example, by executing a machine-readable program or other computer-executable instructions, such as routines, instructions, programs, or other code. The steps, or operations, of the technique 900, or another technique, method, process, or algorithm described in connection with the implementations disclosed herein can be implemented directly in hardware, firmware, software executed by hardware, circuitry, or a combination thereof.

For simplicity of explanation, the technique 900 is depicted and described herein as a series of steps or operations. However, the steps or operations of the technique 900 can occur in various orders and/or concurrently. Additionally, other steps or operations not presented and described herein may be used. Furthermore, not all illustrated steps or operations may be required to implement a technique in accordance with the disclosed subject matter.

At 902, the technique 900 includes obtaining an input record including clinical data associated with a patient encounter. In some implementations, the input record includes at least one of text data, audio data, or video data. At 904, the technique 900 includes providing the input record as input to an ML component configured to perform a RAG ML operation. At 906, the technique 900 includes identifying, by querying a vector database, a set of reference records for use in the RAG ML operation.

In some implementations, the technique 900 includes generating a set of vector embeddings corresponding to the input record, and identifying the set of reference records includes performing a vector similarity operation associated with the set of vector embeddings and at least one additional set of vector embeddings, stored in the vector database, corresponding to the set of reference records. In some implementations, the technique 900 includes determining a set of structured data record metadata associated with the input record, and identifying the set of reference records includes identifying at least one reference record based on the set of structured data record metadata. In some implementations, identifying the set of reference records includes searching, based on a set of structured data record metadata associated with the input record, a vector database to identify a set of structured data record identifier, and determining, based on a context filter, a subset of the set of structured data record identifiers, where the subset of the set of structured data record identifiers corresponds to the set of reference records.

At 908, the technique 900 includes generating, using the machine learning component and based on the input record and the set of reference records, a structured data record associated with the patient encounter. At 910, the technique 900 includes outputting structured record information, corresponding to the structured data record to cause a display of a user device to present a representation of the structured data record.

In some implementations, the technique 900 includes outputting the structured data record for use by an ML-based service engine system in performing one or more retrieval-augmented machine learning operations. In some implementations, the one or more retrieval-augmented machine learning operations includes a diagnosis classification code prediction operation. In some implementations, the one or more retrieval-augmented machine learning operations includes a procedure classification code prediction operation.

In some implementations, the technique 900 includes providing the structured data record to a clinician interface; receiving, via the clinician interface, clinician feedback associated with the structured data record; generating, based on the clinician feedback, a modified structured data record; and outputting the modified structured data record for use by an ML-based service engine system in performing one or more retrieval-augmented machine learning operations. In some implementations, the technique 900 includes providing the structured data record for output to an electronic health record.

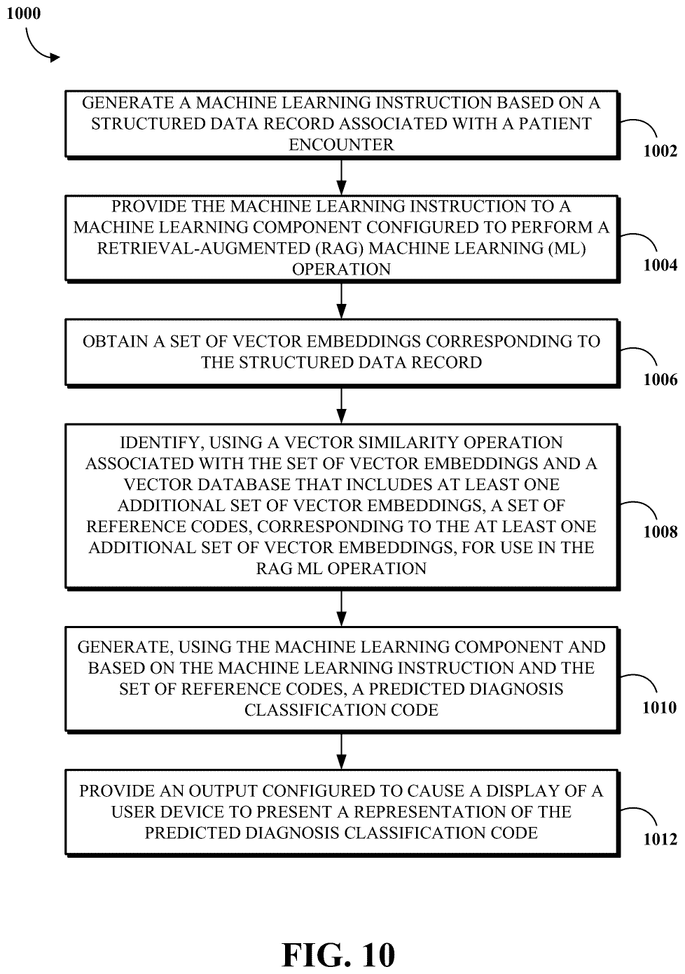

FIG. 10 is a flowchart of an example of a technique 1000 associated with a machine-learning-based clinical workflow. The technique 1000 can be executed using computing devices, such as the systems, hardware, and software described with respect to FIGS. 1-7. The technique 1000 can be performed, for example, by executing a machine-readable program or other computer-executable instructions, such as routines, instructions, programs, or other code. The steps, or operations, of the technique 1000, or another technique, method, process, or algorithm described in connection with the implementations disclosed herein can be implemented directly in hardware, firmware, software executed by hardware, circuitry, or a combination thereof.

For simplicity of explanation, the technique 1000 is depicted and described herein as a series of steps or operations. However, the steps or operations of the technique 1000 can occur in various orders and/or concurrently. Additionally, other steps or operations not presented and described herein may be used. Furthermore, not all illustrated steps or operations may be required to implement a technique in accordance with the disclosed subject matter.

At 1002, the technique 1000 includes generating a machine learning instruction based on a structured data record associated with a patient encounter. At 1004, the technique 1000 includes providing the machine learning instruction to an ML component configured to perform a RAG ML operation.

At 1006, the technique 1000 includes obtaining a set of vector embeddings corresponding to the structured data record. At 1008, the technique 1000 includes identifying, using a vector similarity operation associated with the set of vector embeddings and a vector database that includes at least one additional set of vector embeddings, a set of reference codes, corresponding to the at least one additional set of vector embeddings, for use in the RAG ML operation. At 1010, the technique 1000 includes generating, using the machine learning component and based on the machine learning instruction and the set of reference codes, a predicted diagnosis classification code. At 1012, the technique 1000 includes providing an output configured to cause a display of a user device to present a representation of the predicted diagnosis classification code.

In some implementations, the technique 1000 includes receiving, from the user device, feedback associated with the predicted diagnosis classification code, and updating the machine learning component based on the feedback. In some implementations, the vector database includes sets of embedding vectors associated with at least one of a medical coding system or an electronic health record system. In some implementations, the vector database includes sets of embedding vectors associated with at least one of a code description, a medical guideline, a code exclusion, a synonym, or a disease name.

In some implementations, the predicted diagnosis classification code is based on at least one of a billing code, a diagnostic code, a procedural code, or facility information.

In some implementations, providing the output includes providing the output to a clinician interface, and the technique 1000 further includes receiving, from the clinician interface, feedback associated with the predicted diagnosis classification code, and updating, based on the feedback, a set of code weights associated with the set of reference codes.

In some implementations, the predicted diagnosis classification code includes at least one of a primary diagnosis code or a primary procedure code. In some implementations, the predicted diagnosis classification code is an ICD-10 code. In some implementations, the predicted diagnosis classification code includes at least one of a CPT code or an HCPCS code.

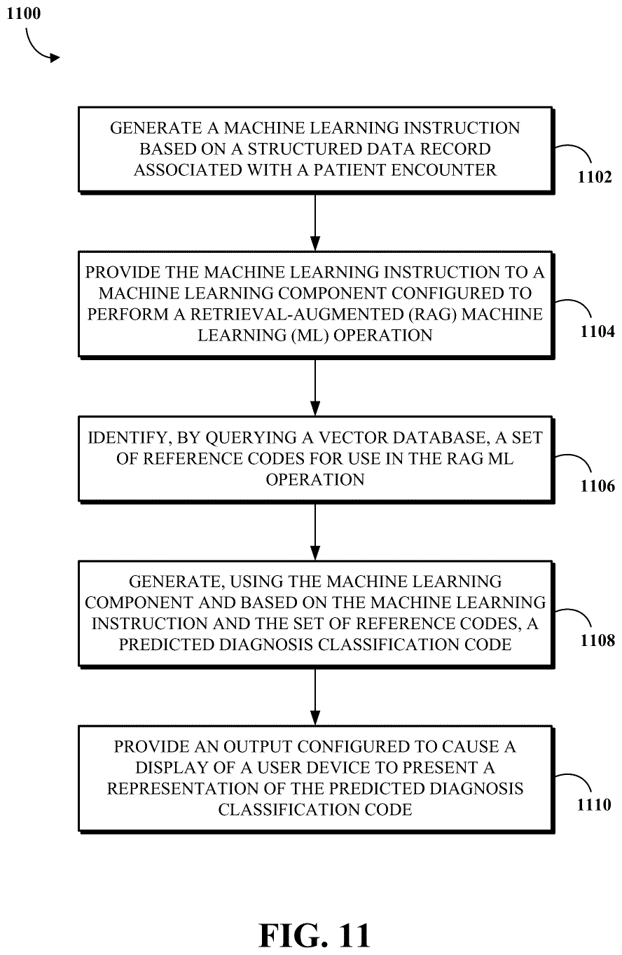

FIG. 11 is a flowchart of an example of a technique 1100 associated with a machine-learning-based clinical workflow. The technique 1100 can be executed using computing devices, such as the systems, hardware, and software described with respect to FIGS. 1-7. The technique 1100 can be performed, for example, by executing a machine-readable program or other computer-executable instructions, such as routines, instructions, programs, or other code. The steps, or operations, of the technique 1100, or another technique, method, process, or algorithm described in connection with the implementations disclosed herein can be implemented directly in hardware, firmware, software executed by hardware, circuitry, or a combination thereof.

For simplicity of explanation, the technique 1100 is depicted and described herein as a series of steps or operations. However, the steps or operations of the technique 1100 can occur in various orders and/or concurrently. Additionally, other steps or operations not presented and described herein may be used. Furthermore, not all illustrated steps or operations may be required to implement a technique in accordance with the disclosed subject matter.

At 1102, the technique 1100 includes generating a machine learning instruction based on a structured data record associated with a patient encounter. At 1104, the technique 1100 includes providing the machine learning instruction to an ML component configured to perform a RAG ML operation. At 1106, the technique 1100 includes identifying, by querying a vector database, a set of reference codes for use in the RAG ML operation. At 1108, the technique 1100 includes generating, using the machine learning component and based on the machine learning instruction and the set of reference codes, a predicted diagnosis classification code. At 1110, the technique 1100 includes providing an output configured to cause a display of a user device to present a representation of the predicted diagnosis classification code.

In some implementations, providing the output includes providing the output to a clinician interface, and the technique 1100 includes receiving, from the clinician interface, feedback associated with the predicted diagnosis classification code, and updating, based on the feedback, a set of code weights associated with the set of reference codes. In some implementations, the predicted diagnosis classification code includes at least one of a primary diagnosis code or a primary procedure code. In some implementations, the predicted diagnosis classification code is an ICD-10 code. In some implementations, the predicted diagnosis classification code includes at least one of a CPT code or an HCPCS code.

In some implementations, the technique 1100 includes receiving, from the user device, feedback associated with the predicted diagnosis classification code, and updating the machine learning component based on the feedback. In some implementations, the vector database includes sets of embedding vectors associated with at least one of a medical coding system or an electronic health record system. In some implementations, the vector database includes sets of embedding vectors associated with at least one of a code description, a medical guideline, a code exclusion, a synonym, or a disease name. In some implementations, the predicted diagnosis classification code is based on at least one of a billing code, a diagnostic code, a procedural code, or facility information.

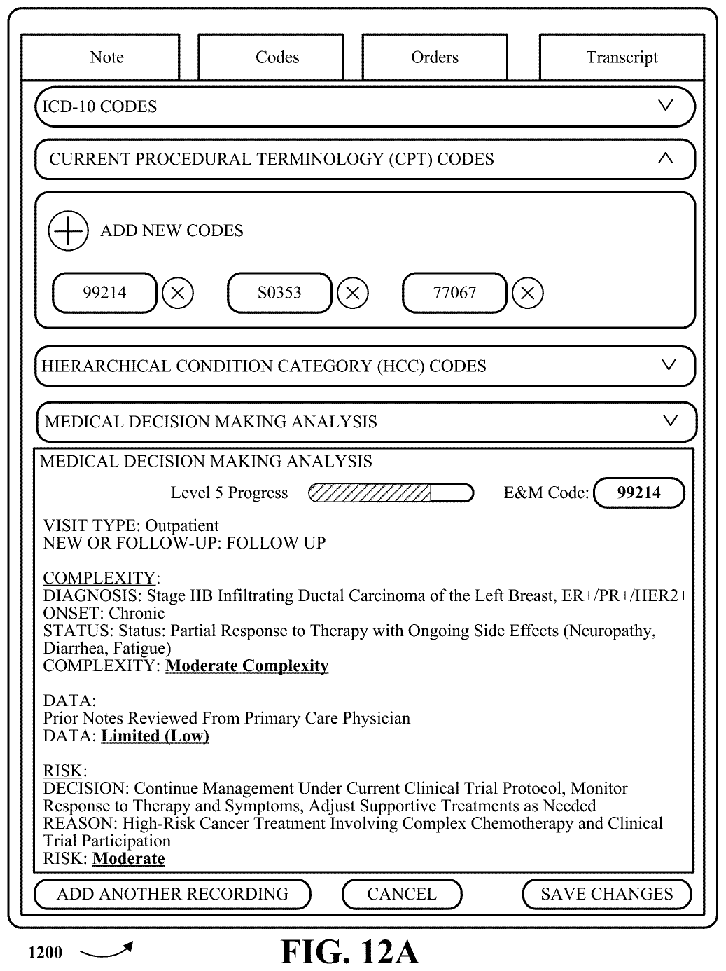

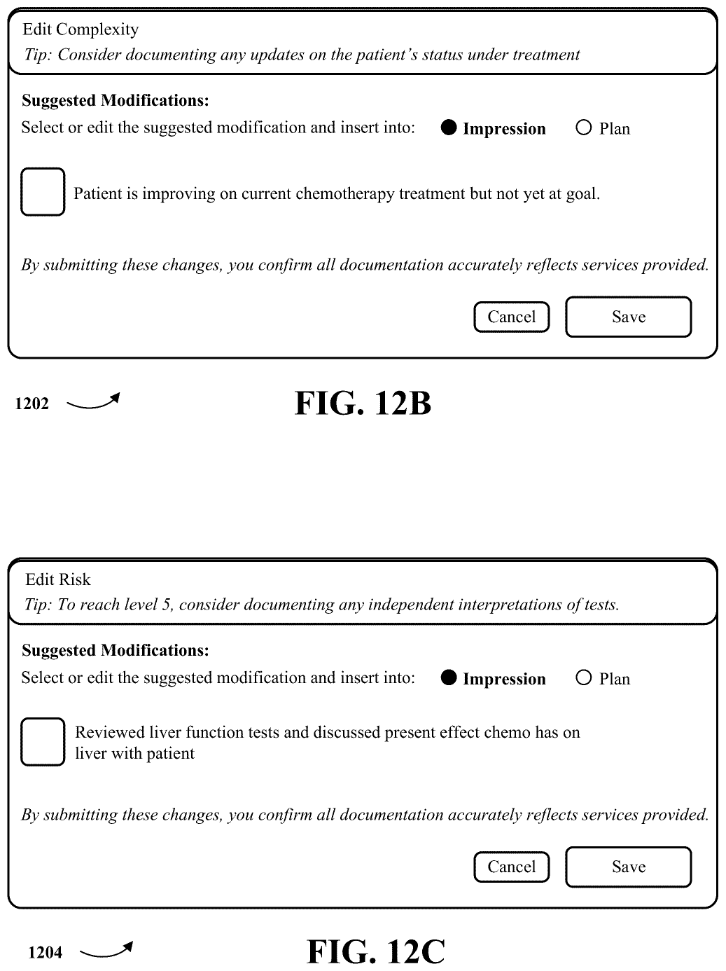

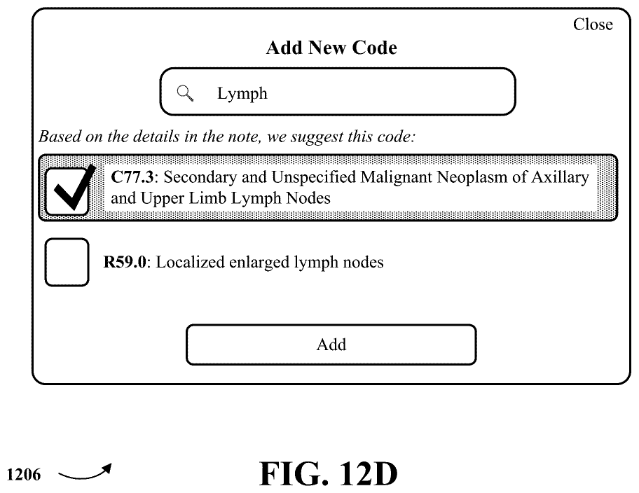

FIGS. 12A-12D illustrate example interfaces 1200, 1202, 1204, and 1206, respectively, for displaying and managing medical coding information in a clinical workflow system, as described herein. The interfaces demonstrate how some implementations may present coding suggestions, allow for code review and modification, and facilitate documentation updates to support optimal code selection. Any one or more of the interfaces 1200, 1202, 1204, or 1206 may be presented on a display of a user device (e.g., the user device 404 shown in FIG. 4) based on output received from a clinical workflow platform (e.g., the clinical workflow platform 402 shown in FIG. 4).

FIG. 12A shows an example interface 1200 with multiple sections for managing different types of medical codes. The interface includes navigation tabs for accessing different sections including Note, Codes, Orders, and Transcript. The navigation tabs may provide access to different aspects of the clinical workflow, organizing information in a way that allows healthcare providers to efficiently manage patient encounters. For example, the Note section may include the clinical documentation interface where providers can view and edit patient encounter notes, including subjective information, objective findings, assessment details, and treatment plans. This section may also display suggested documentation improvements and allow for real-time editing of clinical notes.

The Codes section may provide access to various coding interfaces, including ICD-10 diagnostic codes, CPT procedure codes, and HCC codes. This section may display code suggestions based on the documentation, allow for code search and selection, and provide coding guidance to support appropriate code selection. The interface 1200 may show code descriptions, documentation requirements, and related coding guidelines.

The Orders section may present interfaces for managing medical orders, prescriptions, and referrals. This section may include order templates, medication ordering tools, and laboratory or imaging test ordering capabilities. The interface 1200 may display order sets specific to different conditions or specialties and may incorporate clinical decision support features to guide appropriate order selection.

The Transcript section may provide access to original encounter documentation, such as audio recordings or transcribed notes from patient visits. This section may allow providers to review raw documentation, verify accuracy of transcriptions, and access historical records of patient encounters. The interface 1200 may include audio playback controls, transcription editing tools, and options for annotating or flagging specific portions of the documentation.

Organizing these functions into separate tabs may help reduce cognitive load by presenting focused interfaces for specific tasks while maintaining easy access to related information. This approach may allow healthcare providers to efficiently switch between different aspects of the documentation and coding workflow while maintaining context about the patient encounter. The tab-based navigation may also facilitate workflow customization, as different specialties or practice settings may configure which tabs are most relevant for their specific needs. In some implementations, the system may maintain state across tabs, allowing information entered in one section to automatically update relevant fields in other sections. For example, documentation added in the Note section may trigger updates to code suggestions in the Codes section, while orders placed in the Orders section may automatically generate appropriate procedure codes.

In some implementations, each section of the interface 1200 may include a dropdown menu that reveals additional options and information when selected. For example, the ICD-10 CODES section may expand to display subcategories of diagnostic codes, allowing clinicians to navigate through different code families or specialties. The CPT CODES section may include dropdown options for different procedure types, such as evaluation and management (E&M codes) codes, surgical procedures, or diagnostic tests.

In some implementations, the system may be particularly effective at predicting and suggesting appropriate E&M codes, which may significantly improve revenue capture for healthcare providers. E&M codes represent a complex subset of medical billing codes that reflect the level of complexity and time involved in patient encounters. Accurate E&M coding may be important for proper reimbursement, as undercoding may result in lost revenue while overcoding may lead to compliance issues and potential audits.

The system may analyze various factors such as the complexity of medical decision-making, the amount of data reviewed, and the level of risk associated with the patient's condition to suggest the most appropriate E&M code. By leveraging machine learning algorithms and a comprehensive database of reference codes, the system may identify nuances in the clinical documentation that human coders might overlook, potentially capturing higher-level E&M codes when justified by the documented care.

In some aspects, the progress bar displayed in the interface may provide real-time feedback on the completeness of documentation required for different E&M code levels. This visual representation may allow clinicians to quickly assess whether their current documentation supports the suggested E&M code or if additional information is needed to justify a higher-level code. The progress bar may update dynamically as the clinician adds or modifies information in the clinical note, providing immediate guidance on how close they are to meeting the criteria for the next higher E&M code level.

The progress bar may be advantageous in several ways. It may serve as a prompt for clinicians to document all relevant aspects of the patient encounter, potentially reducing instances of undercoding due to incomplete documentation. By visually indicating the proximity to the next code level, the progress bar may encourage clinicians to provide more comprehensive documentation when clinically appropriate, which may lead to more accurate coding and improved revenue capture. Additionally, the progress bar may help educate clinicians about E&M coding requirements over time, potentially improving their documentation practices and coding accuracy in future encounters.

In some implementations, the system may provide specific suggestions for additional documentation that could support a higher E&M code level. These suggestions may be based on the current content of the clinical note, the patient's medical history, and common elements that are often missing in documentation for higher-level E&M codes. By guiding clinicians to include all relevant information, the system may help ensure that the level of service provided is accurately reflected in the coding, potentially leading to more appropriate reimbursement and reduced risk of revenue leakage.

The ADD NEW CODES area may incorporate dropdown functionality to filter or sort codes based on various criteria such as frequency of use, specialty relevance, or recent updates. When adding codes, clinicians may access dropdown menus that display suggested codes based on the note content, with options to view alternative codes or related documentation requirements. The ADD NEW CODES area may provide several advantages for managing medical coding workflows. In some implementations, the area may display recently added or frequently used codes in a compact format, allowing clinicians to quickly verify their code selections. The removal options associated with each code entry may enable efficient correction of coding decisions without navigating through multiple menus or screens.

The system may implement intelligent code suggestion features in the ADD NEW CODES area. For example, when a clinician begins typing a code or description, the system may display relevant suggestions based on the current patient context, historical coding patterns, and vector similarity matches from the reference database. This approach may help reduce coding errors and improve efficiency. In some implementations, the ADD NEW CODES area may include visual indicators that reflect the confidence level or validation status of each code. These indicators may help clinicians identify codes that may require additional documentation or review. The system may also display relationships between selected codes, helping to identify potential conflicts or opportunities for more specific code selection.

The area may support batch operations for managing multiple codes simultaneously. For example, clinicians may be able to group related codes, apply common modifications, or remove multiple codes with a single action. The interface 1200 may also provide undo/redo functionality for code addition and removal operations, allowing clinicians to easily correct mistakes or experiment with different coding combinations. In some implementations, the ADD NEW CODES area may integrate with the system's machine learning capabilities to provide real-time feedback on code selection. As codes are added or removed, the system may update documentation suggestions, risk scores, or compliance indicators. The area may also display relevant coding guidelines or documentation requirements specific to the selected codes. The system may maintain a historical record of code additions and removals within the ADD NEW CODES area. This history may be used to analyze coding patterns, identify training opportunities, or support audit requirements. The interface 1200 may allow clinicians to view and restore previous code selections when needed.

The HCC CODES section may include expandable subsections for different risk categories, condition groups, or demographic factors. The MEDICAL DECISION MAKING ANALYSIS section may feature dropdown elements that reveal detailed breakdowns of complexity factors, data reviewed, and risk assessments. These expandable sections may display progress indicators, documentation requirements, and suggested modifications to support optimal code selection. In some implementations, dropdown menus may also provide access to additional features such as code history, usage statistics, or reference materials. The interface 1200 may allow clinicians to customize which dropdown options appear by default based on their preferences or specialty requirements.

The medical decision making analysis portion of interface 1200 provides detailed information about the complexity and requirements for different coding levels. For example, it displays a Level 5 Progress indicator with a progress bar, showing how close the current documentation is to qualifying for a higher level of service. The E&M Code field shows code 99214, while additional fields display visit type and follow-up information. The illustrated interface 1200, for example, presents complexity assessment details for a breast cancer case, along with data review information and risk assessment details.

In some implementations, the interface 1200 may incorporate alternative navigation and selection mechanisms to provide flexible access to coding information and functionality. For example, the interface 1200 may include a tree view structure that displays hierarchical relationships between different code categories, allowing clinicians to expand and collapse branches to explore related codes and documentation requirements. The interface 1200 may implement accordion panels that expand vertically when selected, providing a space-efficient way to display detailed information while maintaining context. These panels may automatically adjust their size based on content, helping to optimize screen space usage while presenting comprehensive coding information.

In some implementations, the interface 1200 may utilize tabbed panels that allow horizontal navigation between different code sections. Each tab may contain related information and functionality, enabling clinicians to quickly switch between different aspects of the coding workflow while maintaining their current context. The system may implement a ribbon interface that organizes frequently used functions into logical groups. This approach may provide quick access to common coding tasks while making additional features discoverable through contextual tabs that appear based on the current task or selection. In some implementations, the interface 1200 may include a command palette that responds to keyboard shortcuts or text commands. This feature may allow clinicians to quickly search for and execute coding-related actions without navigating through menus. The command palette may support natural language queries and provide fuzzy matching to help users find relevant commands even with partial or inexact input.

The interface 1200 may incorporate expandable cards or tiles that can be rearranged on the screen according to user preference. These cards may display summary information when collapsed and expand to show detailed options and controls when selected. Users may customize the layout by dragging and dropping cards to create personalized workflows. In some implementations, the system may provide a contextual sidebar that dynamically updates based on the selected content or current task. This sidebar may display relevant reference materials, related codes, or documentation requirements without requiring navigation away from the main interface. The sidebar may be collapsible to maximize screen space when not needed. The interface 1200 may implement a floating action menu that appears near the cursor when content is selected, providing quick access to common actions like adding codes, viewing references, or accessing documentation templates. This context-sensitive menu may adapt its options based on the type of content selected and the current state of the workflow.

In some implementations, the system may utilize gesture-based interactions for touch-enabled devices. These gestures may include swipe actions to navigate between sections, pinch-to-zoom for detailed code information, or multi-finger taps to access contextual menus. The gesture system may be customizable to accommodate different user preferences and device capabilities. The interface 1200 may incorporate voice commands and natural language processing to allow hands-free navigation and code selection. Clinicians may use voice input to search for codes, navigate sections, or document patient encounters. The voice interface may support medical terminology and adapt to individual speaking patterns over time.

The system may generate the interface 1200 through a dynamic process that leverages the machine learning and vector database components to provide real-time, context-aware coding assistance. In some implementations, the system may analyze the structured data record using vector embeddings to identify relevant sections and content that should be displayed. The interface 1200 may adapt its layout and displayed information based on the specialty, user preferences, and specific details of the patient encounter. For example, when processing an oncology case, the system may automatically surface relevant ICD-10 codes and CPT codes commonly used in cancer care while suppressing codes that may be irrelevant to oncology practice.

The system may employ a multi-stage approach to populate the interface elements. In some aspects, the vector similarity operations may identify relevant reference codes and documentation examples that inform the suggested modifications presented to clinicians. The progress indicators and complexity assessments may be generated through continuous analysis of the documentation against coding requirements, with the machine learning component evaluating multiple factors to determine the appropriate service level.

The interface 1200 may incorporate adaptive learning capabilities that allow it to refine its presentations based on user interactions. In some implementations, the system may track which suggestions clinicians commonly accept or reject and adjust its recommendation algorithms accordingly. The dropdown menus and filtering options may be dynamically populated based on historical usage patterns and the current context of the patient encounter. This approach may help reduce cognitive load by presenting the most relevant options first while still maintaining access to the full range of coding possibilities.

The system may generate documentation suggestions by combining information from multiple sources, including the vector database, reference codes, and historical patterns of documentation. In some cases, the interface 1200 may present these suggestions with associated confidence scores or supporting evidence, allowing clinicians to make informed decisions about accepting or modifying the suggested changes. The interface 1200 may also provide real-time feedback as clinicians make modifications, updating progress indicators and complexity assessments to reflect the impact of documentation changes on code selection.

FIGS. 12B and 12C illustrate example interfaces 1202 and 1204 for editing medical documentation to support optimal coding. Interface 1202 focuses on editing complexity documentation, providing a tip suggesting documentation of patient status updates under treatment. The interface 1202 presents a suggested text modification stating “Patient is improving on current chemotherapy treatment but not yet at goal.” This modification may help substantiate the level of medical decision-making complexity required for the selected code.

Interface 1204 demonstrates the system's ability to guide documentation improvements for risk assessment. The interface 1204 provides a tip about documenting independent interpretations of tests to reach level 5 coding requirements. The suggested text modification reads “Reviewed liver function tests and discussed present effect chemo has on liver with patient.” Both interfaces 1202 and 1204 include radio button options for “Impression” and “Plan” sections, allowing clinicians to specify where the modifications should be inserted.

The system may leverage several machine learning techniques to generate these documentation suggestions and interface elements. In some implementations, the system may employ a retrieval-augmented generation approach that combines the strengths of large language models with vector similarity search capabilities. This approach may allow the system to identify relevant historical cases and documentation examples that closely match the current clinical scenario.

The vector embeddings generated from clinical notes and documentation may capture complex relationships between medical concepts, procedures, and outcomes. In some aspects, these embeddings may enable the system to understand the contextual significance of different documentation elements and their relationship to specific code requirements. For example, when suggesting documentation improvements for complexity or risk assessment, the system may analyze vector representations of similar cases to identify patterns in how experienced clinicians document similar scenarios.

The machine learning component may utilize a multi-task learning architecture that simultaneously considers multiple objectives when generating suggestions. These objectives may include maximizing documentation completeness, ensuring compliance with coding requirements, and maintaining consistency with the clinician's documentation style. This approach may help produce suggestions that are both technically accurate and naturally integrated into the clinician's workflow.

In some implementations, the system may incorporate attention mechanisms that allow the machine learning models to focus on the most relevant parts of the input data when generating suggestions. These mechanisms may help the system identify key phrases or concepts in the clinical documentation that may need elaboration or clarification to support specific code levels.

The system's ability to provide real-time feedback may be enhanced by incremental learning capabilities that allow the models to continuously adapt to new patterns and preferences. As clinicians interact with the suggested modifications, the system may update its internal representations and adjust its suggestion strategies. This adaptive approach may help improve the relevance and usefulness of suggestions over time.

The machine learning components may also implement uncertainty quantification techniques that help determine the confidence level of different suggestions. In some aspects, this uncertainty information may be used to prioritize suggestions and determine how they are presented in the interface. Suggestions with higher confidence scores may be displayed more prominently, while those with lower confidence may be presented as optional alternatives.

FIG. 12D shows an example interface 1206 for adding diagnostic codes through an intelligent search function. The interface displays a search field containing “Lymph” and presents suggested diagnostic codes based on the note details. The system shows two relevant options: a selected code “C77.3: Secondary and Unspecified Malignant Neoplasm of Axillary and Upper Limb Lymph Nodes” and an unselected code “R59.0: Localized enlarged lymph nodes.” This demonstrates the system's ability to suggest specific, relevant codes based on the clinical documentation.

The interfaces incorporate several innovative features that help prevent revenue leakage and improve coding accuracy. For example, the progress bar in interface 1200 provides real-time feedback on documentation completeness, helping clinicians identify opportunities to support higher-level codes. The suggested text modifications in interfaces 1202 and 1204 guide clinicians in documenting the necessary elements to substantiate their code selections, reducing the risk of denials or downcoding.

In some implementations, interfaces (e.g., the interface 1200) may include features for analyzing the financial impact of different coding decisions. In some implementations, the system may display estimated reimbursement information alongside suggested codes, helping clinicians understand the revenue implications of their documentation and coding choices. The system may also track historical reimbursement patterns and denial rates for specific code combinations, using this information to suggest alternative coding strategies that may optimize revenue capture while maintaining compliance.

The interface may provide visualization tools that help clinicians track their documentation and coding performance over time. In some implementations, these tools may display trends in code selection, denial rates, and revenue capture, allowing clinicians to identify areas where additional attention to documentation may help prevent revenue leakage. The system may also benchmark individual performance against peer groups or organizational standards, providing context for identifying improvement opportunities.

The machine learning components may be configured to identify complex cases or unusual clinical scenarios that may require special attention to documentation and coding. In some aspects, the system may flag these cases for additional review and provide enhanced documentation guidance to ensure appropriate revenue capture. The system may also analyze patterns in successful appeals of denied claims, using this information to strengthen documentation suggestions for similar cases in the future.

The system's ability to dynamically suggest documentation improvements based on selected codes represents a significant advance over traditional coding workflows. Rather than requiring clinicians to memorize complex coding requirements, the interfaces provide contextual guidance and specific suggestions for documentation updates. This helps ensure that the clinical documentation accurately reflects the complexity and scope of services provided.

The interfaces demonstrate particular value for specialty-specific workflows, such as oncology practice. By suggesting specific, detailed ICD-10 codes like C77.3 instead of general codes, the system helps practices capture the full complexity of patient conditions. The integration of E&M level analysis with suggested documentation improvements helps practices optimize reimbursement while maintaining compliance with coding guidelines.

The system may provide specialized support for various medical specialties through customized documentation templates, code suggestions, and workflow optimizations. For example, in cardiology practices, the system may automatically highlight relevant sections of electrocardiogram (ECG) reports and suggest appropriate Current Procedural Terminology (CPT) codes for different types of cardiac monitoring. The system may also provide cardiology-specific documentation templates that include fields for ejection fraction measurements, valve assessments, and other cardiac-specific parameters.

In orthopedic settings, the system may incorporate anatomical diagrams and body-part specific documentation tools. The interface may allow clinicians to quickly document range of motion measurements, strength assessments, and surgical planning details. The system may suggest relevant ICD-10 codes based on the specific joint or body part being examined, potentially improving the accuracy and specificity of diagnosis coding.

For dermatology practices, the system may include features for documenting lesion locations, sizes, and characteristics. The interface may provide specialized templates for procedures such as biopsies and excisions, automatically suggesting appropriate modifiers based on the anatomical location and size of the lesion. The system may maintain a database of dermatology-specific terminology and common diagnostic patterns to enhance code suggestion accuracy.

In psychiatric practices, the system may offer templates designed to capture mental status examinations, behavioral observations, and therapeutic interventions. The interface may provide structured fields for documenting time-based services and may suggest appropriate evaluation and management codes based on the complexity of the psychiatric assessment and treatment planning.

For obstetrics and gynecology practices, the system may include specialized workflows for tracking pregnancy progression and managing preventive care services. The interface may provide trimester-specific documentation templates and may suggest appropriate diagnostic codes based on the gestational age and any identified complications. The system may also track and suggest preventive service codes based on patient age and risk factors.

In pediatric settings, the system may incorporate age-specific developmental assessments and growth tracking tools. The interface may provide templates for well-child visits that align with standard developmental milestones and immunization schedules. The system may suggest age-appropriate preventive service codes and may help identify opportunities for additional screening services based on patient risk factors.

For emergency medicine, the system may provide rapid documentation tools optimized for fast-paced environments. The interface may include quick-select templates for common emergency presentations and may suggest appropriate evaluation and management codes based on the documented level of service and medical decision making. The system may also help track critical care time and other time-based services.

In neurology practices, the system may offer specialized templates for documenting neurological examinations and cognitive assessments. The interface may provide structured fields for recording detailed neurological findings and may suggest appropriate diagnostic codes based on the documented neurological signs and symptoms. The system may also help track the progression of neurological conditions over time.

For pulmonology practices, the system may incorporate tools for documenting pulmonary function tests and respiratory assessments. The interface may provide templates for recording detailed breathing parameters and may suggest appropriate diagnostic codes based on the documented respiratory findings. The system may also help track the management of chronic respiratory conditions.

The system may also adapt to subspecialty-specific requirements within these broader specialties. For example, in interventional cardiology, the system may provide detailed documentation templates for cardiac catheterization procedures. In pediatric endocrinology, the system may offer specialized growth charts and metabolic disorder tracking tools.

According to an aspect of the present disclosure, a method is provided. The method includes obtaining an input record comprising clinical data associated with a patient encounter. The method includes providing the input record as input to a machine learning component configured to perform a retrieval-augmented (RAG) machine learning (ML) operation. The method includes generating a set of vector embeddings corresponding to the input record. The method includes identifying, using a vector similarity operation associated with the set of vector embeddings and a vector database that includes at least one additional set of vector embeddings, a set of reference records, corresponding to the at least one additional set of vector embeddings, for use in the RAG ML operation. The method includes generating, using the machine learning component and based on the input record and the set of reference records, a structured data record associated with the patient encounter. The method includes outputting structured record information, corresponding to the structured data record to cause a display of a user device to present a representation of the structured data record.

According to other aspects of the present disclosure, the method may include one or more of the following features. The input record may comprise at least one of text data, audio data, or video data. The method may include outputting the structured data record for use by an ML-based service engine system in performing one or more retrieval-augmented machine learning operations. The one or more retrieval-augmented machine learning operations may include a diagnosis classification code prediction operation. The one or more retrieval-augmented machine learning operations may include a procedure classification code prediction operation. The one or more retrieval-augmented machine learning operations may include an order generation operation. The method may include determining a set of structured data record metadata associated with the input record, wherein identifying the set of reference records comprises identifying at least one reference record based on the set of structured data record metadata. The method may include searching, based on a set of structured data record metadata associated with the input record, a vector database to identify a set of structured data record identifiers, and determining, based on a context filter, a subset of the set of structured data record identifiers, wherein the subset of the set of structured data record identifiers corresponds to the set of reference records. The method may include providing the structured data record to a clinician interface, receiving, via the clinician interface, clinician feedback associated with the structured data record, generating, based on the clinician feedback, a modified structured data record, and outputting the modified structured data record for use by an ML-based service engine system in performing one or more retrieval-augmented machine learning operations. The method may include providing the structured data record for output to an electronic health record.

According to another aspect of the present disclosure, a system is provided. The system includes a memory subsystem storing instructions and a processing system configured to execute the instructions to cause the system to obtain an input record comprising clinical data associated with a patient encounter, provide the input record as input to a machine learning component configured to perform a retrieval-augmented (RAG) machine learning (ML) operation, identify, by querying a vector database, a set of reference records for use in the RAG ML operation, generate, using the machine learning component and based on the input record and the set of reference records, a structured data record associated with the patient encounter, and output structured record information, corresponding to the structured data record to cause a display of a user device to present a representation of the structured data record.

According to other aspects of the present disclosure, the system may include one or more of the following features. The processing system may be configured to execute the instructions to further cause the system to generate a set of vector embeddings corresponding to the input record, wherein, to identify the set of reference records, the processing system is configured to execute the instructions to cause the system to perform a vector similarity operation associated with the set of vector embeddings and at least one additional set of vector embeddings, stored in the vector database, corresponding to the set of reference records. The input record may comprise at least one of text data, audio data, or video data. The processing system may be configured to execute the instructions to further cause the system to output the structured data record for use by an ML-based service engine system in performing one or more retrieval-augmented machine learning operations. The one or more retrieval-augmented machine learning operations may include a diagnosis classification code prediction operation. The one or more retrieval-augmented machine learning operations may include a procedure classification code prediction operation. The processing system may be configured to execute the instructions to further cause the system to determine a set of structured data record metadata associated with the input record, and wherein, to identify the set of reference records, the processing system is configured to execute the instructions to cause the system to identify at least one reference record based on the set of structured data record metadata. The processing system may be configured to execute the instructions to further cause the system to search, based on a set of structured data record metadata associated with the input record, a vector database to identify a set of structured data record identifiers, and determine, based on a context filter, a subset of the set of structured data record identifiers, wherein the subset of the set of structured data record identifiers corresponds to the set of reference records. The processing system may be configured to execute the instructions to further cause the system to provide the structured data record to a clinician interface, receive, via the clinician interface, clinician feedback associated with the structured data record, generate, based on the clinician feedback, a modified structured data record, and output the modified structured data record for use by an ML-based service engine system in performing one or more retrieval-augmented machine learning operations. The processing system may be configured to execute the instructions to further cause the system to provide the structured data record for output to an electronic health record.

According to another aspect of the present disclosure, a non-transitory computer readable medium storing instructions operable to cause one or more processors to perform operations is provided. The operations include obtaining an input record comprising clinical data associated with a patient encounter, providing the input record as input to a machine learning component, generating, using the machine learning component and based on the input record and a set of reference records, a structured data record associated with the patient encounter, and outputting structured record information, corresponding to the structured data record to cause a display of a user device to present a representation of the structured data record.

According to other aspects of the present disclosure, the operations may include one or more of the following features. The operations may include generating a set of vector embeddings corresponding to the input record, and identifying the set of reference records using a vector similarity operation associated with the set of vector embeddings and a vector database that includes at least one additional set of vector embeddings corresponding to the set of reference records. The input record may comprise at least one of text data, audio data, or video data. The operations may include outputting the structured data record for use by an ML-based service engine system in performing one or more retrieval-augmented machine learning operations. The one or more retrieval-augmented machine learning operations may include a diagnosis classification code prediction operation. The one or more retrieval-augmented machine learning operations may include a procedure classification code prediction operation. The operations may include determining a set of structured data record metadata associated with the input record, wherein identifying the set of reference records comprises identifying at least one reference record based on the set of structured data record metadata. The operations may include searching, based on a set of structured data record metadata associated with the input record, a vector database to identify a set of structured data record identifiers, and determining, based on a context filter, a subset of the set of structured data record identifiers, wherein the subset of the set of structured data record identifiers corresponds to the set of reference records. The operations may include providing the structured data record to a clinician interface, receiving, via the clinician interface, clinician feedback associated with the structured data record, generating, based on the clinician feedback, a modified structured data record, and outputting the modified structured data record for use by an ML-based service engine system in performing one or more retrieval-augmented machine learning operations. The operations may include providing the structured data record for output to an electronic health record.

According to another aspect of the present disclosure, a method is provided. The method includes generating a machine learning instruction based on a structured data record associated with a patient encounter, providing the machine learning instruction to a machine learning component configured to perform a retrieval-augmented (RAG) machine learning (ML) operation, obtaining a set of vector embeddings corresponding to the structured data record, identifying, using a vector similarity operation associated with the set of vector embeddings and a vector database that includes at least one additional set of vector embeddings, a set of reference codes, corresponding to the at least one additional set of vector embeddings, for use in the RAG ML operation, generating, using the machine learning component and based on the machine learning instruction and the set of reference codes, a predicted diagnosis classification code, and providing an output configured to cause a display of a user device to present a representation of the predicted diagnosis classification code.

According to other aspects of the present disclosure, the method may include one or more of the following features. The method may include receiving, from the user device, feedback associated with the predicted diagnosis classification code, and updating the machine learning component based on the feedback. The vector database may comprise sets of embedding vectors associated with at least one of a medical coding system or an electronic health record system. The vector database may comprise sets of embedding vectors associated with at least one of a code description, a medical guideline, a code exclusion, a synonym, or a disease name. The predicted diagnosis classification code may be based on at least one of a billing code, a diagnostic code, a procedural code, or facility information. The method may include providing the output to a clinician interface, receiving, from the clinician interface, feedback associated with the predicted diagnosis classification code, and updating, based on the feedback, a set of code weights associated with the set of reference codes. The predicted diagnosis classification code may comprise at least one of a primary diagnosis code or a primary procedure code. The predicted diagnosis classification code may be an ICD-10 code. The predicted diagnosis classification code may comprise at least one of a current procedural terminology (CPT) code or a healthcare common procedure coding system (HCPCS) code.

According to another aspect of the present disclosure, a system is provided. The system includes a memory subsystem storing instructions and a processing system configured to execute the instructions to cause the system to generate a machine learning instruction based on a structured data record associated with a patient encounter, provide the machine learning instruction to a machine learning component configured to perform a retrieval-augmented (RAG) machine learning (ML) operation, identify, by querying a vector database, a set of reference codes for use in the RAG ML operation, generate, using the machine learning component and based on the machine learning instruction and the set of reference codes, a predicted diagnosis classification code, and provide an output configured to cause a display of a user device to present a representation of the predicted diagnosis classification code.

According to other aspects of the present disclosure, the system may include one or more of the following features. The processing system may be configured to execute the instructions to further cause the system to receive, from the user device, feedback associated with the predicted diagnosis classification code, and update the machine learning component based on the feedback. The vector database may comprise sets of embedding vectors associated with at least one of a medical coding system or an electronic health record system. The vector database may comprise sets of embedding vectors associated with at least one of a code description, a medical guideline, a code exclusion, a synonym, or a disease name. The predicted diagnosis classification code may be based on at least one of a billing code, a diagnostic code, a procedural code, or facility information. The processing system may be configured to execute the instructions to cause the system to provide the output to a clinician interface, receive, from the clinician interface, feedback associated with the predicted diagnosis classification code, and update, based on the feedback, a set of code weights associated with the set of reference codes. The predicted diagnosis classification code may comprise at least one of a primary diagnosis code or a primary procedure code. The predicted diagnosis classification code may be an ICD-10 code. The predicted diagnosis classification code may comprise at least one of a current procedural terminology (CPT) code or a healthcare common procedure coding system (HCPCS) code.

According to another aspect of the present disclosure, a non-transitory computer readable medium storing instructions operable to cause one or more processors to perform operations is provided. The operations include generating a machine learning instruction based on a structured data record associated with a patient encounter, providing the machine learning instruction to a machine learning component, generating, using the machine learning component and based on the machine learning instruction and a set of reference codes, a predicted diagnosis classification code, and providing an output configured to cause a display of a user device to present a representation of the predicted diagnosis classification code.

According to other aspects of the present disclosure, the operations may include one or more of the following features. The operations may include obtaining a set of vector embeddings corresponding to the structured data record, and identifying the set of reference codes using a vector similarity operation associated with the set of vector embeddings and a vector database that includes at least one additional set of vector embeddings corresponding to the set of reference codes. The operations may include receiving, from the user device, feedback associated with the predicted diagnosis classification code, and updating the machine learning component based on the feedback. The predicted diagnosis classification code may be based on at least one of a billing code, a diagnostic code, a procedural code, or facility information. The operations may include providing the output to a clinician interface, receiving, from the clinician interface, feedback associated with the predicted diagnosis classification code, and updating, based on the feedback, a set of code weights associated with the set of reference codes. The predicted diagnosis classification code may comprise at least one of a primary diagnosis code or a primary procedure code. The predicted diagnosis classification code may be an ICD-10 code. The predicted diagnosis classification code may comprise at least one of a current procedural terminology (CPT) code or a healthcare common procedure coding system (HCPCS) code.

Aspect 1: A method involves obtaining an input record containing clinical data from a patient encounter, providing this record to a machine learning component configured for retrieval-augmented machine learning operations, generating vector embeddings corresponding to the input record, identifying reference records using vector similarity operations between these embeddings and additional embeddings stored in a vector database, generating a structured data record using the machine learning component based on both the input record and reference records, and outputting structured record information to display a representation of the structured data record on a user device.

Aspect 2: The method of Aspect 1, where the input record includes at least one of text data, audio data, or video data.

Aspect 3: The method of either of Aspects 1 or 2, additionally involving outputting the structured data record for use by a machine learning-based service engine system in performing one or more retrieval-augmented machine learning operations.

Aspect 4: The method of Aspect 3, where the retrieval-augmented machine learning operations include a diagnosis classification code prediction operation.

Aspect 5: The method of either of Aspects 3 or 4, where the retrieval-augmented machine learning operations include a procedure classification code prediction operation.

Aspect 6: The method of any of Aspects 3-5, where the retrieval-augmented machine learning operations include an order generation operation.

Aspect 7: The method of any of Aspects 1-6, additionally involving determining structured data record metadata associated with the input record, where identifying the reference records includes identifying at least one reference record based on the structured data record metadata.

Aspect 8: The method of any of Aspects 1-7, where identifying the reference records involves searching a vector database based on structured data record metadata to identify structured data record identifiers, and determining a subset of these identifiers based on a context filter, where this subset corresponds to the reference records.

Aspect 9: The method of any of Aspects 1-8, additionally involving providing the structured data record to a clinician interface, receiving clinician feedback about the structured data record through this interface, generating a modified structured data record based on the feedback, and outputting the modified record for use by a machine learning-based service engine system in performing retrieval-augmented machine learning operations.

Aspect 10: The method of any of Aspects 1-9, additionally involving providing the structured data record for output to an electronic health record.

Aspect 11: A system comprising a memory subsystem storing instructions and a processing system configured to execute these instructions to obtain an input record containing clinical data from a patient encounter, provide this record to a machine learning component configured for retrieval-augmented machine learning operations, identify reference records by querying a vector database, generate a structured data record using the machine learning component based on both the input record and reference records, and output structured record information to display a representation of the structured data record on a user device.

Aspect 12: The system of Aspect 11, where the processing system is configured to execute instructions to generate vector embeddings corresponding to the input record, and identify reference records by performing vector similarity operations between these embeddings and additional embeddings stored in the vector database that correspond to the reference records.

Aspect 13: The system of either of Aspects 11 or 12, where the input record includes at least one of text data, audio data, or video data.

Aspect 14: The system of any of Aspects 11-13, where the processing system is configured to execute instructions to output the structured data record for use by a machine learning-based service engine system in performing retrieval-augmented machine learning operations.

Aspect 15: The system of Aspect 14, where the retrieval-augmented machine learning operations include a diagnosis classification code prediction operation.

Aspect 16: The system of either of Aspects 14 or 15, where the retrieval-augmented machine learning operations include a procedure classification code prediction operation.

Aspect 17: The system of any of Aspects 11-16, where the processing system is configured to execute instructions to determine structured data record metadata associated with the input record and identify reference records based on this metadata.

Aspect 18: The system of any of Aspects 11-17, where the processing system is configured to execute instructions to identify reference records by searching a vector database based on structured data record metadata to identify structured data record identifiers, and determining a subset of these identifiers based on a context filter, where this subset corresponds to the reference records.

Aspect 19: The system of any of Aspects 11-18, where the processing system is configured to execute instructions to provide the structured data record to a clinician interface, receive clinician feedback about the structured data record through this interface, generate a modified structured data record based on the feedback, and output the modified record for use by a machine learning-based service engine system in performing retrieval-augmented machine learning operations.

Aspect 20: The system of any of Aspects 11-19, where the processing system is configured to execute instructions to provide the structured data record for output to an electronic health record.

Aspect 21: A non-transitory computer readable medium storing instructions that cause processors to perform operations including obtaining an input record containing clinical data from a patient encounter, providing this record to a machine learning component, generating a structured data record using the machine learning component based on both the input record and reference records, and outputting structured record information to display a representation of the structured data record on a user device.

Aspect 22: The non-transitory computer readable medium of Aspect 21, where the operations additionally include generating vector embeddings corresponding to the input record and identifying reference records using vector similarity operations between these embeddings and additional embeddings stored in a vector database that correspond to the reference records.

Aspect 23: The non-transitory computer readable medium of either of Aspects 21 or 22, where the input record includes at least one of text data, audio data, or video data.

Aspect 24: The non-transitory computer readable medium of any of Aspects 21-23, where the operations additionally include outputting the structured data record for use by a machine learning-based service engine system in performing retrieval-augmented machine learning operations.

Aspect 25: The non-transitory computer readable medium of Aspect 24, where the retrieval-augmented machine learning operations include a diagnosis classification code prediction operation.

Aspect 26: The non-transitory computer readable medium of either of Aspects 24 or 25, where the retrieval-augmented machine learning operations include a procedure classification code prediction operation.

Aspect 27: The non-transitory computer readable medium of any of Aspects 21-26, where the operations additionally include determining structured data record metadata associated with the input record and identifying reference records based on this metadata.

Aspect 28: The non-transitory computer readable medium of any of Aspects 21-27, where identifying reference records involves searching a vector database based on structured data record metadata to identify structured data record identifiers, and determining a subset of these identifiers based on a context filter, where this subset corresponds to the reference records.

Aspect 29: The non-transitory computer readable medium of any of Aspects 21-28, where the operations additionally include providing the structured data record to a clinician interface, receiving clinician feedback about the structured data record through this interface, generating a modified structured data record based on the feedback, and outputting the modified record for use by a machine learning-based service engine system in performing retrieval-augmented machine learning operations.

Aspect 30: The non-transitory computer readable medium of any of Aspects 21-29, where the operations additionally include providing the structured data record for output to an electronic health record.

Aspect 31: A method involves generating a machine learning instruction based on a structured data record from a patient encounter, providing this instruction to a machine learning component configured for retrieval-augmented machine learning operations, obtaining vector embeddings corresponding to the structured data record, identifying reference codes using vector similarity operations between these embeddings and additional embeddings stored in a vector database, generating a predicted diagnosis classification code using the machine learning component based on both the instruction and reference codes, and providing output to display a representation of the predicted code on a user device.

Aspect 32: The method of Aspect 31, additionally involving receiving feedback about the predicted diagnosis classification code from the user device and updating the machine learning component based on this feedback.

Aspect 33: The method of either of Aspects 31 or 32, where the vector database contains embedding vectors associated with at least one of a medical coding system or an electronic health record system.

Aspect 34: The method of any of Aspects 31-33, where the vector database contains embedding vectors associated with at least one of a code description, medical guideline, code exclusion, synonym, or disease name.

Aspect 35: The method of any of Aspects 31-34, where the predicted diagnosis classification code is based on at least one of a billing code, diagnostic code, procedural code, or facility information.

Aspect 36: The method of any of Aspects 31-35, where providing output involves providing it to a clinician interface and additionally involves receiving feedback about the predicted diagnosis classification code through this interface and updating code weights associated with the reference codes based on this feedback.

Aspect 37: The method of any of Aspects 31-36, where the predicted diagnosis classification code includes at least one of a primary diagnosis code or a primary procedure code.

Aspect 38: The method of any of Aspects 31-37, where the predicted diagnosis classification code is an ICD-10 code.

Aspect 39: The method of any of Aspects 31-38, where the predicted diagnosis classification code includes at least one of a current procedural terminology (CPT) code or a healthcare common procedure coding system (HCPCS) code.

Aspect 40: A system comprising a memory subsystem storing instructions and a processing system configured to execute these instructions to generate a machine learning instruction based on a structured data record from a patient encounter, provide this instruction to a machine learning component configured for retrieval-augmented machine learning operations, identify reference codes by querying a vector database, generate a predicted diagnosis classification code using the machine learning component based on both the instruction and reference codes, and provide output to display a representation of the predicted code on a user device.

Aspect 41: The system of Aspect 40, where the processing system is configured to execute instructions to receive feedback about the predicted diagnosis classification code from the user device and update the machine learning component based on this feedback.

Aspect 42: The system of either of Aspects 40 or 41, where the vector database contains embedding vectors associated with at least one of a medical coding system or an electronic health record system.

Aspect 43: The system of Aspect 42, where the vector database contains embedding vectors associated with at least one of a code description, medical guideline, code exclusion, synonym, or disease name.

Aspect 44: The system of either of Aspects 42 or 43, where the predicted diagnosis classification code is based on at least one of a billing code, diagnostic code, procedural code, or facility information.

Aspect 45: The system of any of Aspects 40-44, where the processing system is configured to execute instructions to provide output to a clinician interface, receive feedback about the predicted diagnosis classification code through this interface, and update code weights associated with the reference codes based on this feedback.

Aspect 46: The system of any of Aspects 40-45, where the predicted diagnosis classification code includes at least one of a primary diagnosis code or a primary procedure code.

Aspect 47: The system of any of Aspects 40-46, where the predicted diagnosis classification code is an ICD-10 code.

Aspect 48: The system of any of Aspects 40-47, where the predicted diagnosis classification code includes at least one of a current procedural terminology (CPT) code or a healthcare common procedure coding system (HCPCS) code.

Aspect 49: A non-transitory computer readable medium storing instructions that cause processors to perform operations including generating a machine learning instruction based on a structured data record from a patient encounter, providing this instruction to a machine learning component, generating a predicted diagnosis classification code using the machine learning component based on both the instruction and reference codes, and providing output to display a representation of the predicted code on a user device.

Aspect 50: The non-transitory computer readable medium of Aspect 49, where the operations additionally include obtaining vector embeddings corresponding to the structured data record and identifying reference codes using vector similarity operations between these embeddings and additional embeddings stored in a vector database that correspond to the reference codes.

Aspect 51: The non-transitory computer readable medium of either of Aspects 49 or 50, where the operations additionally include receiving feedback about the predicted diagnosis classification code from the user device and updating the machine learning component based on this feedback.

Aspect 52: The non-transitory computer readable medium of any of Aspects 49-51, where the predicted diagnosis classification code is based on at least one of a billing code, diagnostic code, procedural code, or facility information.

Aspect 53: The non-transitory computer readable medium of any of Aspects 49-52, where providing output involves providing it to a clinician interface and the operations additionally include receiving feedback about the predicted diagnosis classification code through this interface and updating code weights associated with the reference codes based on this feedback.

Aspect 54: The non-transitory computer readable medium of any of Aspects 49-53, where the predicted diagnosis classification code includes at least one of a primary diagnosis code or a primary procedure code.

Aspect 55: The non-transitory computer readable medium of any of Aspects 49-54, where the predicted diagnosis classification code is an ICD-10 code.

Aspect 56: The non-transitory computer readable medium of any of Aspects 49-55, where the predicted diagnosis classification code includes at least one of a current procedural terminology (CPT) code or a healthcare common procedure coding system (HCPCS) code.

As used herein, the term “component” is intended to be broadly construed as hardware and/or a combination of hardware and software. “Software” shall be construed broadly to mean instructions, instruction sets, code, code segments, program code, programs, subprograms, software modules, applications, software applications, software packages, routines, subroutines, objects, executables, threads of execution, procedures, and/or functions, among other examples, whether referred to as software, firmware, middleware, microcode, hardware description language, or otherwise. As used herein, a processor is implemented in hardware and/or a combination of hardware and software. It will be apparent that systems and/or methods described herein may be implemented in different forms of hardware and/or a combination of hardware and software. The actual specialized control hardware or software code used to implement these systems and/or methods is not limiting of the aspects. Thus, the operation and behavior of the systems and/or methods were described herein without reference to specific software code—it being understood that software and hardware can be designed to implement the systems and/or methods based, at least in part, on the description herein.

As used herein, satisfying a threshold may, depending on the context, refer to a value being greater than the threshold, greater than or equal to the threshold, less than the threshold, less than or equal to the threshold, equal to the threshold, not equal to the threshold, or the like.

Even though particular combinations of features are recited in the claims and/or disclosed in the specification, these combinations are not intended to limit the disclosure of various aspects. In fact, many of these features may be combined in ways not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the terms “set” and “group” are intended to include one or more items (e.g., related items, unrelated items, or a combination of related and unrelated items), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

The adjectives “first,” “second,” “third,” and so on are used for contextual distinction between two or more of the modified nouns in connection with a discussion and are not meant to be absolute modifiers that apply only to a certain respective node throughout the entire document. For example, a component may be referred to as a “first component” in connection with one discussion and may be referred to as a “second component” in connection with another discussion, or vice versa. Reference to a component, a computing device, a server, a client, an application, an apparatus, a device, a system, a computing system, or the like may include disclosure of the computing device, server, client, application, apparatus, device, system, computing system, or the like, respectively, being a node. For example, disclosure that a computing device is configured to receive information from a server also discloses that a first node is configured to receive information from a second node. Consistent with this disclosure, once a specific example is broadened in accordance with this disclosure (e.g., a computing device is configured to receive information from a server also discloses that a first node is configured to receive information from a second node), the broader example of the narrower example may be interpreted in the reverse, but in a broad open-ended way. In the example above where a computing device being configured to receive information from a server also discloses a first node being configured to receive information from a second node, “first node” may refer to a first computing device, a first server, a first client, a first application, a first apparatus, a first device, a first system, a first computing system, or the like, configured to receive the information from a second node; and “second node” may refer to a second computing device, a second server, a second client, a second application, a second apparatus, a second device, a second system, a second computing system, or the like.

As used herein, unless explicitly stated otherwise, any term specified in the singular may include its plural version. For example, “a computer that stores data and runs software,” may include a single computer that stores data and runs software or two computers-a first computer that stores data and a second computer that runs software. Also “a computer that stores data and runs software,” may include multiple computers that together stored data and run software. At least one of the multiple computers stores data, and at least one of the multiple computers runs software.

As used herein, the term “computer-readable medium” encompasses one or more computer readable media. A computer-readable medium may include any storage unit (or multiple storage units) that store data or instructions that are readable by a processing system. A computer-readable medium may include, for example, at least one of a data repository, a data storage unit, a computer memory, a hard drive, a disk, or a random access memory. A computer-readable medium may include a single computer-readable medium or multiple computer-readable media. A computer-readable medium may be a transitory computer-readable medium or a non-transitory computer-readable medium.

As used herein, the term “memory subsystem” includes one or more memories, where each memory may be a computer-readable medium. A memory subsystem may encompass memory hardware units (e.g., a hard drive or a disk) that store data or instructions in software form. Alternatively or in addition, the memory subsystem may include data or instructions that are hard-wired into processing system.

A processor may include one or more chips, system-on-chips (SoCs), chipsets, packages, or devices that individually or collectively constitute or comprise a processing system. The processing system includes a processor (or “processing”) circuitry in the form of one or multiple processors, microprocessors, processing units (such as central processing units (CPUs), graphics processing units (GPUs), neural processing units (NPUs) and/or digital signal processors (DSPs)), processing blocks, application-specific integrated circuits (ASIC), programmable logic devices (PLDs) (such as field programmable gate arrays (FPGAs)), or other discrete gate or transistor logic or circuitry (all of which may be generally referred to herein individually as “processors” or collectively as “the processor” or “the processor circuitry”). One or more of the processors may be individually or collectively configurable or configured to perform various functions or operations described herein. A group of processors collectively configurable or configured to perform a set of functions may include a first processor configurable or configured to perform a first function of the set and a second processor configurable or configured to perform a second function of the set, or may include the group of processors all being configured or configurable to perform the set of functions.

The processing system may further include memory circuitry in the form of one or more memory devices, memory blocks, memory elements or other discrete gate or transistor logic or circuitry, each of which may include tangible storage media such as random-access memory (RAM) or read-only memory (ROM), or combinations thereof (all of which may be generally referred to herein individually as “memories” or collectively as “the memory” or “the memory circuitry”). One or more of the memories may be coupled (for example, operatively coupled, communicatively coupled, electronically coupled, or electrically coupled) with one or more of the processors and may individually or collectively store processor-executable code (such as software) that, when executed by one or more of the processors, may configure one or more of the processors to perform various functions or operations described herein. Additionally or alternatively, in some examples, one or more of the processors may be preconfigured to perform various functions or operations described herein without requiring configuration by software.

As used herein, the term “engine” may include software, hardware, or a combination of software and hardware. An engine may be implemented using software stored in the memory subsystem. Alternatively, an engine may be hard-wired into the processing system. In some cases, an engine includes a combination of software stored in the memory subsystem and hardware that is hard-wired into the processing system.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “mechanism” and “component” are used broadly and are not limited to mechanical or physical implementations, but can include software routines in conjunction with processors, etc. Likewise, the terms “system” or “tool” as used herein and in the figures, but in any event based on their context, may be understood as corresponding to a functional unit implemented using software, hardware (e.g., an integrated circuit, such as an ASIC), or a combination of software and hardware. In certain contexts, such systems or mechanisms may be understood to be a processor-implemented software system or processor-implemented software mechanism that is part of or callable by an executable program, which may itself be wholly or partly composed of such linked systems or mechanisms.

Implementations or portions of implementations of the above disclosure can take the form of a computer program product accessible from, for example, a computer-usable or computer-readable medium. A computer-usable or computer-readable medium can be a device that can, for example, tangibly contain, store, communicate, or transport a program or data structure for use by or in connection with a processor. The medium can be, for example, an electronic, magnetic, optical, electromagnetic, or semiconductor device.

Other suitable mediums are also available. Such computer-usable or computer-readable media can be referred to as non-transitory memory or media, and can include volatile memory or non-volatile memory that can change over time. The quality of memory or media being non-transitory refers to such memory or media storing data for some period of time or otherwise based on device power or a device power cycle. A memory of an apparatus described herein, unless otherwise specified, does not have to be physically contained by the apparatus, but is one that can be accessed remotely by the apparatus, and does not have to be contiguous with other memory that might be physically contained by the apparatus.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method, comprising:
generating a machine learning instruction based on a structured data record associated with a patient encounter;
providing the machine learning instruction to a machine learning component configured to perform a retrieval-augmented (RAG) machine learning (ML) operation;
obtaining a set of vector embeddings corresponding to the structured data record;
identifying, using a vector similarity operation associated with the set of vector embeddings and a vector database that includes at least one additional set of vector embeddings, a set of reference codes, corresponding to the at least one additional set of vector embeddings, for use in the RAG ML operation;
generating, using the machine learning component and based on the machine learning instruction and the set of reference codes, a predicted diagnosis classification code; and
providing an output configured to cause a display of a user device to present a representation of the predicted diagnosis classification code.

2. The method of claim 1, further comprising:
receiving, from the user device, feedback associated with the predicted diagnosis classification code; and
updating the machine learning component based on the feedback.

3. The method of claim 1, wherein the vector database comprises sets of embedding vectors associated with at least one of a medical coding system or an electronic health record system.

4. The method of claim 1, wherein the vector database comprises sets of embedding vectors associated with at least one of a code description, a medical guideline, a code exclusion, a synonym, or a disease name.

5. The method of claim 1, wherein the predicted diagnosis classification code is based on at least one of a billing code, a diagnostic code, a procedural code, or facility information.

6. The method of claim 1, wherein providing the output comprises:
providing the output to a clinician interface, the method further comprising:
receiving, from the clinician interface, feedback associated with the predicted diagnosis classification code; and
updating, based on the feedback, a set of code weights associated with the set of reference codes.

7. The method of claim 1, wherein the predicted diagnosis classification code comprises at least one of a primary diagnosis code or a primary procedure code.

8. The method of claim 1, wherein the predicted diagnosis classification code is an ICD-10 code.

9. The method of claim 1, wherein the predicted diagnosis classification code comprises at least one of a current procedural terminology (CPT) code or a healthcare common procedure coding system (HCPCS) code.

10. A system, comprising:
a memory subsystem storing instructions; and
a processing system configured to execute the instructions to cause the system to:
generate a machine learning instruction based on a structured data record associated with a patient encounter;
provide the machine learning instruction to a machine learning component configured to perform a retrieval-augmented (RAG) machine learning (ML) operation;
identify, by querying a vector database, a set of reference codes for use in the RAG ML operation;
generate, using the machine learning component and based on the machine learning instruction and the set of reference codes, a predicted diagnosis classification code; and
provide an output configured to cause a display of a user device to present a representation of the predicted diagnosis classification code.

11. The system of claim 10, wherein the processing system is configured to execute the instructions to further cause the system to:
receive, from the user device, feedback associated with the predicted diagnosis classification code; and
update the machine learning component based on the feedback.

12. The system of claim 10, wherein the vector database comprises sets of embedding vectors associated with at least one of a medical coding system or an electronic health record system.

13. The system of claim 12, wherein the vector database comprises sets of embedding vectors associated with at least one of a code description, a medical guideline, a code exclusion, a synonym, or a disease name.

14. The system of claim 12, wherein the predicted diagnosis classification code is based on at least one of a billing code, a diagnostic code, a procedural code, or facility information.

15. The system of claim 10, wherein, to provide the output, the processing system is configured to execute the instructions to cause the system to:
provide the output to a clinician interface, and wherein the processing system is configured to execute the instructions to further cause the system to:
receive, from the clinician interface, feedback associated with the predicted diagnosis classification code; and
update, based on the feedback, a set of code weights associated with the set of reference codes.

16. The system of claim 10, wherein the predicted diagnosis classification code comprises at least one of a primary diagnosis code or a primary procedure code.

17. The system of claim 10, wherein the predicted diagnosis classification code comprises at least one of a current procedural terminology (CPT) code or a healthcare common procedure coding system (HCPCS) code.

18. A non-transitory computer readable medium storing instructions operable to cause one or more processors to perform operations comprising:
generating a machine learning instruction based on a structured data record associated with a patient encounter;
providing the machine learning instruction to a machine learning component;
generating, using the machine learning component and based on the machine learning instruction and a set of reference codes, a predicted diagnosis classification code; and
providing an output configured to cause a display of a user device to present a representation of the predicted diagnosis classification code.

19. The non-transitory computer readable medium of claim 18, the operations further comprising:
obtaining a set of vector embeddings corresponding to the structured data record; and
identifying the set of reference codes using a vector similarity operation associated with the set of vector embeddings and a vector database that includes at least one additional set of vector embeddings corresponding to the set of reference codes.

20. The non-transitory computer readable medium of claim 18, the operations further comprising:
receiving, from the user device, feedback associated with the predicted diagnosis classification code; and
updating the machine learning component based on the feedback.
